(19) United States
(12) Patent Application Publication
(10) Pub. No.: US 2011/0293578 A1
(43) Pub. Date: Dec. 1, 2011

(54) Use of Stem Cells to Prevent Neuronal Dieback

(75) Inventors: Sarah A. Busch, Cleveland, OH (US); Kevin P. Horn, Gahanna, OH (US); Robert W. Mays, Shaker Heights, OH (US); Jerry Silver, Bay Village, OH (US)

(73) Assignees: Case Western Reserve University, Cleveland, OH (US); ABT Holding Company, Cleveland, OH (US)

(21) Appl. No.: 13/062,343

(22) PCT Filed: Sep. 4, 2009
(86) PCT No.: PCT/US09/56046

§ 371 Date: Aug. 18, 2011

Related U.S. Application Data
(60) Provisional application No. 61/094,210, filed on Sep. 4, 2008.

(51) Int. Cl. A61K 35/12; C12N 5/079; A61P 25/16; A61P 25/00; A61P 25/28; A61P 25/08; A61K 38/18; C12N 5/0786

(52) U.S. Cl. 424/93.7; 514/8.3; 435/375

(57) ABSTRACT

The invention is generally directed to treatment of neuronal injury. In particular, the invention is directed to reducing axonal retraction (“dieback”) that occurs as a result of the interaction of activated macrophages with dystrophic axons that are produced during nervous system acute or chronic injury. The invention is also directed to promoting axonal growth/regeneration. The invention is specifically directed to using stem cells or their secreted cellular factors, such as would be produced in conditioned cell culture medium, to ameliorate or prevent axonal dieback and/or promote growth/regeneration of axons.

### FIELD OF THE INVENTION

The invention is generally directed to treatment of neuronal injury. In particular, the invention is directed to reducing axonal retraction (“dieback”) that occurs as a result of the interaction of activated macrophages with dystrophic axons that are produced during nervous system acute or chronic injury. The invention is also directed to promoting axonal growth/regeneration. The invention is specifically directed to using stem cells or their secreted cellular factors, such as would be produced in conditioned cell culture medium, to ameliorate or prevent axonal dieback and/or promote growth/regeneration of axons.

### BACKGROUND OF THE INVENTION

### Axonal Retraction

After spinal cord injury, a glial scar forms that poses a major impediment to CNS regeneration (Silver and Miller, 2004). In the region of forming scar tissue, the ends of the regenerating axons cease extending and become swollen and distorted into various bizarrely shaped “growth cones” that can remain for years within axon tracts (Ramón y Cajal, 1928; Li and Raisman, 1995; Houle and Jin, 2001; Kwon et al., 2002). Injured axons within the CNS withdraw from the site of axotomy during a period of hours to weeks after an initial injury. There have been differing reports as to the nature of axonal retraction, its cause, extent, and timing as well as discussion of whether it is a passive or active process (Fayaz and Tator, 2000).

### In Vitro Glial Scar Model

In the region of forming scar tissue, several classes of growth inhibitory molecules are upregulated, including the family of extracellular matrix (ECM) molecules known as chondroitin/keratan sulfate proteoglycans (PGs) (Fitch and Silver, 1997; Morgenstern et al., 2002; Jones et al., 2003; Tang et al., 2003). PGs are organized in a crude gradient with the lowest concentrations in the lesion penumbra and the highest in the epicenter (Davies et al., 1999; Fitch et al., 1999). The inhibitory ECM components block the potential of reactive glial cells to support axonal regeneration via laminin (McKeon et al., 1991). Microtransplantation experiments show that adult sensory neurons have a robust capacity for regeneration when placed away from the lesion. Once the regenerating fibers reach the vicinity of the injury site, they are capable of struggling into the lesion penumbra but eventually cease extending and become dystrophic as they penetrate deeply into areas of highest PG concentration (Davies et al., 1999; Grimpe and Silver, 2004).

In vitro glial scar models, that used sharp-edged (i.e., stripe) substrate assays to examine the effects of PGs on axons, induced either growth cone turning or collapse, but not dystrophy (Snow et al., 1990). However, in a recent model of the glial scar, a crude gradient of PGs was sufficient to produce dystrophic endings in regenerating adult axons (Tom et al., 2004). This in vitro system forces regenerating axons of adult sensory neurons to cope with a spot gradient of the PG aggrecan mixed with laminin. Bulbous multivesiculated endings were formed in this glial scar model. PGs led to growth cone dystrophy and dynamic dystrophic endings.

### Inflammation and Injury in Neuronal Tissue

The environment of a spinal cord lesion is extremely complex. Components of the glial scar, such as highly sulfated proteoglycans, ephs, slits, and myelin membrane fragments, (Silver and Miller, 2004; Yiu and He, 2006; Busch and Silver, 2007) as well as the process of neuroinflammation (Donnelly and Popovich, 2007) all contribute to regeneration failure. Inflammatory cells accumulate within the lesion (Fitch et al., 1999). Astrocytes move away from the center of the lesion, become hypertrophic, and upregulate production of inhibitory chondroitin sulfate proteoglycans (CSPGs) that, in turn, cause the formation of dystrophic endbulbs on the severed fibers (Tom et al., 2004). Oligodendrocytes within the lesion die, leading to demyelination, which results in high concentrations of inhibitory myelin breakdown products (Yiu and He, 2006; Xie and Zheng, 2008).

While the inhibitory effects of proteoglycans and myelin on axonal growth were well-established, the role of neuroinflammation in regeneration and regeneration failure remained highly controversial (Popovich and Longbrake, 2008). However, studies have indicated that macrophage infiltration results in increased lesion size, decreased growth of regenerating fibers, and increased death of neurons spared by the initial lesion (Fitch et al., 1999; McPhail et al., 2004; Donnelly and Popovich, 2007). The negative effects of activated macrophages and neutrophils are thought to be mediated by the secretion of cytokines, eicosanoids, free radicals, and proteases, which can be toxic to both neurons and glia (Donnelly and Popovich, 2007). Numerous studies in which macrophages have been depleted, inhibited, or inactivated after spinal cord injury have reported neuroprotection, increased regeneration, and improvements in motor, sensory, and autonomic function (Oudega et al., 1999; Popovich et al., 1999; McPhail et al., 2004; Stirling et al., 2004).

### SUMMARY OF THE INVENTION

The invention is based in part on the inventors' observation that, in an in vitro glial scar model, axonal retraction (dieback) ED-1⁺ cells, such as activated macrophages and microglia, can be reduced by the external administration of certain types of cell or conditioned cell culture medium in which the cells were grown. These in vitro results were also confirmed by cells applied in an in vivo spinal cord injury model.

The inventors observed that ED-1⁺ cells, such as activated macrophages and microglia, adhered to dystrophic axons and that this was necessary for retraction. They found that application of the cells, or conditioned medium from the cells, to the dystrophic axons reduced or prevented adhesion. They further found that application of conditioned medium from culturing the cells had neurostimulatory effects and significantly increased neurite outgrowth/regeneration.

Accordingly, the invention is generally directed to a method for treating (ameliorating or preventing) neuronal injury that is associated with axonal retraction.

The invention is generally directed to a method for treating (ameliorating or preventing) neuronal injury by promoting axonal growth/regeneration in or around a lesion.

The invention is also generally directed to a method for reducing axonal retraction in neuronal injury.

The invention is also generally directed to a method for promoting axonal growth/regeneration in or around a lesion.

Retraction can be caused by ED-1⁺ cells, such as activated macrophages and/or microglia.

The invention is also generally directed to a method for reducing adhesion of ED-1⁺ cells to dystrophic axons so as to reduce axonal retraction.

These results are achieved by administering cells in sufficient proximity to the lesion, for a time sufficient, and in sufficient amount to promote axonal growth/regeneration in or around the lesion.

These results are achieved by administering cells in sufficient proximity to the lesion, for a time sufficient, and in sufficient amount to reduce axonal retraction in neuronal injury and reduce neuronal injury that is associated with axonal retraction.

These results are achieved by administering cells in sufficient proximity to the lesion, for time sufficient, and in sufficient amount to reduce the adhesion of ED-1⁺ cells to dystrophic axons, which adhesion would result in axonal retraction.

The cells are introduced to injured axons so that the cells reduce adhesion of resident ED-1⁺ cells to the axons.

ED-1⁺ cells include, but are not limited to, macrophages and microglia.

These results are caused by factors secreted by the cells. Therefore, the results are also achieved by using a cell culture-conditioned medium or fractions thereof or proteins or other factors derived from the conditioned medium. The conditioned medium is produced by growing the cells, that are effective to reduce adhesion and axonal retraction and/or promote axonal growth, in cell culture. In one embodiment, the conditioned medium is not frozen before use.

These results are also achieved using a cell lysate or cellular fractions.

The cells, secreted factors, fractions, etc., disclosed above, may be administered at various timepoints that correspond to axonal retraction and the injury that results from it, such as at the time of an acute injury, to extended periods (e.g., weeks) after the initial acute injury.

It is understood, however, that axonal retraction may also occur in chronic injury conditions, such as those described below. In chronic injury, the cells may be administered according to any regimen that would reduce axonal retraction.

Because the cells (and secreted factors) also promote axonal growth, they also may be administered in injuries, chronic and acute, that are not necessarily associated with retraction. Such injuries are treated so as to provide and promote axonal growth/regeneration in or around the lesion.

In one embodiment, the cells are stem cells. Stem cells include, but are not limited to, embryonic stem cells and non-embryonic stem cells. The non-embryonic stem cells, like embryonic stem cells, may have the ability to differentiate into cell types of more than one embryonic germ layer and/or express one or more markers associated with the potential to differentiate into cell types of more than one embryonic germ layer. Non-embryonic cells also include tissue-specific stem cells, i.e., that have the ability to differentiate into cells types of only one embryonic germ layer, for example, hematopoietic stem cells, neural stem cells, and mesenchymal stem cells.

In a specific embodiment, the non-embryonic stem cells have been designated “multipotent adult progenitor cells” (“MAPC”) and are described in U.S. Pat. No. 7,015,037.

The invention encompasses any nervous system injury that produces axonal dystrophy where ED-1⁺ cells, such as activated macrophages or microglia, interact with the dystrophic axons and cause the axons to retract. This includes tissues of the central nervous system, including brain and spinal cord. Conditions associated with dystrophic axons include, but are not limited to, spinal cord injury produced by any type of traumatic influence to the spinal cord (these include any force coming from outside the spinal cord (including disc herniation)) or coming from within the spinal cord, such as syringomyelia; brain injury (i.e., head trauma) produced by any type of traumatic influence from within or outside the brain; stroke (ischemic or hemolytic) throughout the central nervous system; multiple sclerosis; epilepsy; neurodegenerative diseases, such as Alzheimer's Disease, Parkinson's Disease, amylotropic lateral sclerosis (Lou Gehrig's Disease), and Creutzfeldt-Jakob Disease (CJD).

### BRIEF DESCRIPTION OF THE FIGURES

FIG. 1—Schematic representation of regeneration failure after spinal cord injury.

FIG. 2—Schematic representation of axonal dieback in vivo.

FIG. 3—Actual and graphical representation of macrophage infiltration and axonal dieback following dorsal column crush. Macrophage infiltration correlates with axonal retraction after spinal cord injury. There is extensive retraction of ascending sensory axons over time after spinal cord injury. A, B, Shown are image montages of 20 μm thick longitudinal sections of a dorsal column crush (DCC) spinal cord injury 2 d (A) and 7 d (B) after lesion. Dex-TR, Texas Red conjugated dextran 3000 MW. The orientation of the sections is such that caudal is on the left side of the image and rostral is on the right. The white boxes below represent axonal position with respect to the lesion center (dotted lines) with superimposed fiber tracings of multiple sections from one animal at each time point. The ruler tick marks indicate 200 μm increments. A, At 2 d after lesion, dorsal root ganglion axons (red) have retracted a short distance from the initial site of axotomy at the lesion center, marked by GFAP+ reactive astrocytes (blue). There are a few ED-1+ cells (green) within the lesion, which are most likely activated microglia. B, By 7 d after lesion, injured axons (red) have retracted extensively from the lesion center. The lesion and surrounding tissue are now filled with ED-1+ cells (green), which are predominantly infiltrating macrophages, whereas reactive astrocytes (blue) have vacated the lesion core. C, Graph indicating average axonal retraction over time. The majority of retraction occurred during the first 7 d after lesion; however, retraction did continue up to 28 d after lesion, the length of time studied. Axonal retraction (black graph) is as follows: day 2 is significantly different from days 7, 14, 28 (one-way ANOVA, F(4,40)=6.50, p<0.001; Tukey's post hoc test, #p<0.05, *p<0.01, **p<0.001). Macrophage depletion (red graph) is as follows: day 2 is significant from days 7, 14, and 28; day 4 is significant from 14 and 28; day 7 is significant from days 2 and 14 (one-way ANOVA, F(4,40)=22.83, p<0.001; Tukey's post hoc test, *p<0.01, **p<0.001,***p<0.0001). Error bars indicate SEM. Scale bars: A, B, 250 μm.

FIG. 4—Time-lapse montage of macrophages inducing neuronal dieback in vitro. Macrophages induce extensive retraction of dystrophic adult dorsal root ganglion axons in an in vitro model of the glial scar. A, Six-panel montage of single-frame images from a time-lapse movie in which NR8383 macrophages were added to a culture of dystrophic adult dorsal root ganglion neurons growing on an inverse spot gradient of the growth-promoting extracellular matrix molecule laminin and the potently inhibitory chondroitin sulfate proteoglycan aggrecan. Times for each frame are given in the bottom right of each image, and an arrow marks the central domain of the growth cone. An asterisk marks a consistent point in the culture as a reference for growth cone position during frame shifts. Initial macrophage-growth cone contact was made immediately after macrophage addition at 30 min. Physical contacts are observed between a second macrophage and the dystrophic axon at 61 min. Additional macrophages physically altered the axonal trajectory as retraction began at 110 min. The growth cone is obscured by multiple macrophages and has retracted nearly out of the frame at 150 min. Scale bar, 20 μm. B, Positional graph tracking the growth cone for entire time-lapse movie in A. Each point represents the position of the central domain of the growth cone for a single frame (every 30 s). The axon underwent extensive retraction of ˜100 μm after macrophage contact. C, Positional graph from another representative time-lapse experiment.

FIG. 5—Contacts formed between axons and macrophages. Macrophages physically interact with dystrophic axons in an in vitro model of the glial scar. A, Select frames from a time-lapse movie in which macrophages physically contact a dystrophic axon. Before retraction occurred, the growth cone was still attached while the axon was lifted from the substrate and severely bent (arrows). B, A higher magnification image of the third image from FIG. 3A. Several adhesive contacts were made between a macrophage and a dystrophic axon. The arrows indicate membrane processes that formed from these contacts as the macrophage moved away from the axon. C, A 40× confocal z-stack three-dimensional reconstruction of a culture of adult DRG neurons (red) 2.5 h after macrophage (green) addition. A macrophage is observed in direct contact with the dystrophic growth cone. D, A 90° rotation of C about the x-axis yielding a side view of the three-dimensional reconstruction. The arrow indicates a neuronal process (red) that has been lifted from the substrate by the adjacent macrophage (green). Scale bars: A, B, 20 μm; C, 50 μm.

FIG. 6—Time-lapse montage of MMP9 inhibitor preventing axonal dieback from macrophage contact.

FIG. 7—Experimental design to assess the effect of externally-added living cells (MAPCs) or conditioned medium on macrophage-induced dorsal root ganglion (DRG) neuron dieback.

FIG. 8—Time-lapse montage of MAPCs co-cultured with DRGs showing that the addition of MAPCs prevent macrophage-induced dieback. MAPCs are administered one day before the addition of macrophages.

FIG. 9—Time-lapse montage of experiment showing that MAPC-conditioned medium prevents macrophage-induced axonal dieback. Conditioned medium is added thirty minutes prior to the addition of macrophages.

FIG. 10—Time-lapse montage showing that macrophages stimulated with MAPC-conditioned medium do not induce axonal dieback.

FIG. 11—Graphical representation of MAPCs preventing macrophage-mediated axonal dieback.

FIG. 12—Experimental summary of in vitro experiments with MAPC or conditioned medium

FIG. 13—MAPCs prevent macrophage-mediated axonal dieback after dorsal column crush injury and promote regeneration into the lesion core. Graphical and actual representation of seven day post-injury spinal cord sections in which a vehicle control or MAPCs were transplanted. Macrophages induce extensive retraction of dystrophic adult dorsal root ganglion axons in an in vitro model of the glial scar. NR838 macrophages were added to a culture of dystrophic adult dorsal root ganglion neurons growing on an inverse spot gradient of the growth-promoting extracellular matrix molecule laminin and the potently inhibitory chondroitin sulfate proteoglycan aggrecan. A positional graph tracks the growth cone for entire time-lapse movie. Each point represents the position of the central domain of the growth cone for a single frame (every 30 s). The axons underwent extensive retraction of ˜100 μm after macrophage contact.

The panels show a 10× image montages of 20 μm thick longitudinal sections of a dorsal column crush (DCC) spinal cord injury 7 d after lesion. Fibers are labeled with Texas Red-conjugated 3000 MW dextran and macrophages are visualized with ED-1+ (purple). The orientation of the sections is such that caudal is on the left side of the image and rostral is on the right. The lesion center is marked below (solid black lines) with three superimposed fiber tracings of multiple sections from one animal for each condition. A, At 7 days after lesion and vehicle injection only, dorsal root ganglion axons (red) have retracted extensively distance from the initial site of axotomy at the lesion center. B, By 7 d after lesion and MAPC transplant, injured axons have regenerated into the lesion in large numbers. C, Graph indicating average axonal retraction over 2, 4, and 7 days after injury in animals receiving vehicle control or MAPC transplants. The conditions, MAPC transplant versus Vehicle control, are significantly different from each other by General Linear Model, *p<0.0001. Scale Bar: A, B, 200 μm.

FIG. 14—Time-lapse montage showing that NG2⁺ glial cells do not prevent macrophage-induced axonal retraction. NG2+ cells stabilize axons, but do not prevent macrophage-mediated retraction following macrophage attack in vitro. A, 40× confocal image showing the association of axons of beta-tubulin+ (red) axons with NG2+ (green) cells on a gradient of aggrecan and laminin after 2 days in vitro. The rim is denoted by a white dotted line. B, NG2+ cells express vimentin (red). C, NG2+ cells express nestin (red). D, Six representative frames from a time-lapse movie illustrating macrophage/axon interactions on an aggrecan/laminin gradient in the presence of adult mouse spinal cord NG2+ cells. NR8383 macrophages are added to a 2 DIV culture of adult DRG neurons. Times for each frame are given in the lower right of each image and an asterisk marks a consistent point on the culture dish as a reference for position during frame shifts. An arrow denotes the central domain of the grown cone. Macrophages are added following a 30 minute period of observation and first contact occurs at 103′. The axon has already undergone a long distance retraction by 110′. Open arrow indicates the presence of a retraction fiber. E, Graph of growth cone position for each frame (30 sec) of the time lapse movie shown in D. Red arc represents the location of the inner rim of the spot. Arrow indicates initial trajectory of growth. F, Distance from the origin of six dystrophic axons in co-culture with NG2+ cells on the aggrecan/laminin spot gradient following contact with macrophages. An arrowhead indicates the position at which the axon has retracted to an NG2 cell. Scale Bar: A, B, C, 50 μm. D, 20 μm.

FIG. 15—Confocal image of MAPCs cultured on a spot gradient alone and higher magnification image of MAPCs growing with neurons on the spot gradient.

10× confocal image of GFP+ MAPC (green) cultured on a bidirectional gradient of aggrecan, visualized by CS56 (red), and laminin 40× confocal image of MAPC co-cultured with adult DRG neurons visualized by β-tubulin (blue). Both adult DRGs and MAPCs do not cross the inhibitory spot rim after 2 days in vitro.

MAPCs added to the aggrecan spot gradient did not invade the inhibitory rim, but adhered well the center of the spot and associated with adult DRG axons.

FIG. 16—Graphical and actual representation of the effect of control media or MAPC-conditioned media on axon outgrowth in vitro.

FIG. 17—Time-lapse montage of experiment showing that control medium does not prevent macrophage-induced axonal dieback. Conditioned medium is added thirty minutes prior to the addition of macrophages.

Macrophages induce extensive retraction of dystrophic adult dorsal root ganglion axons in an in vitro model of the glial scar despite the presence of control MAPC media. A, Six-panel montage of single-frame images from a time-lapse movie in which NR8383 macrophages were added to a culture of dystrophic adult dorsal root ganglion neurons growing on an inverse spot gradient of the growth-promoting extracellular matrix molecule laminin and the potently inhibitory chondroitin sulfate proteoglycan aggrecan. Times for each frame are given in the bottom right of each image, and an arrow marks the central domain of the growth cone. An asterisk marks a consistent point in the culture as a reference for growth cone position during frame shifts. Scale bar, 20 μm. B, Positional graph tracking the growth cone for entire time-lapse movie in A. Each point represents the position of the central domain of the growth cone for a single frame (every 30 s). The axon underwent extensive retraction of ˜80 μm after macrophage contact.

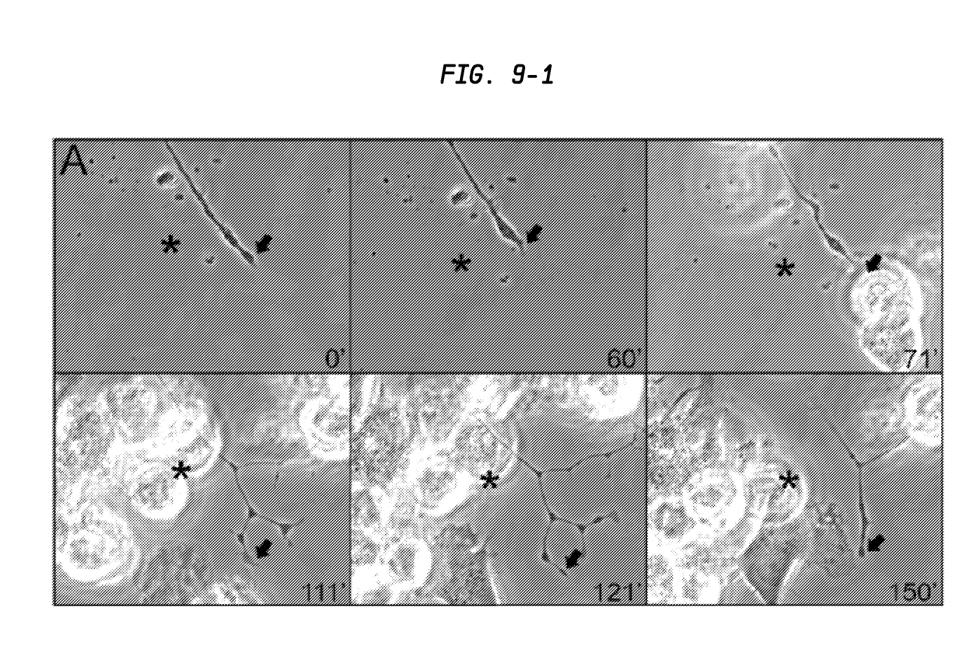

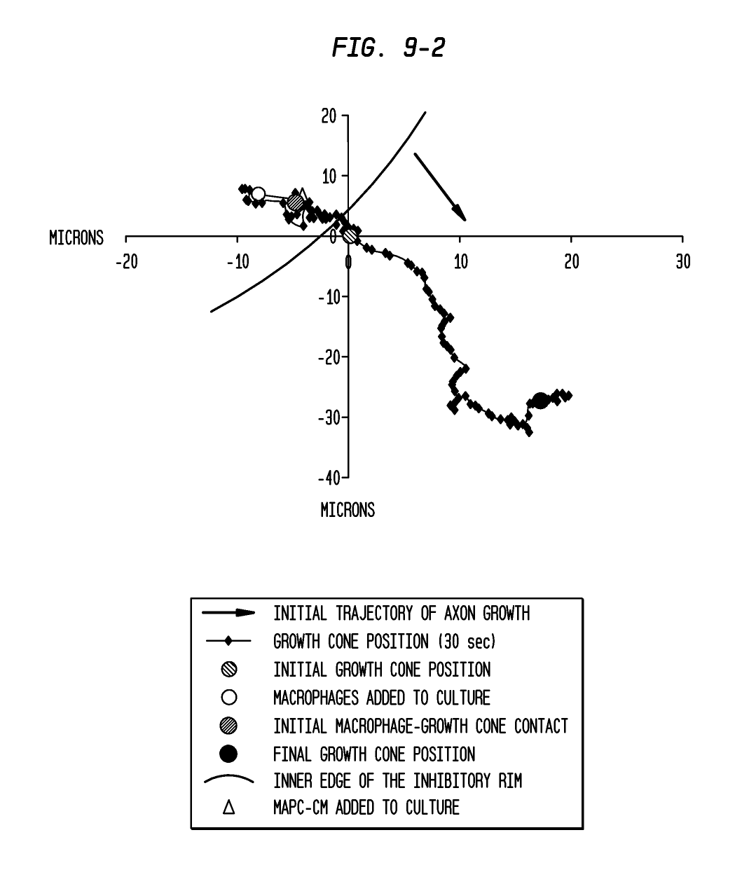

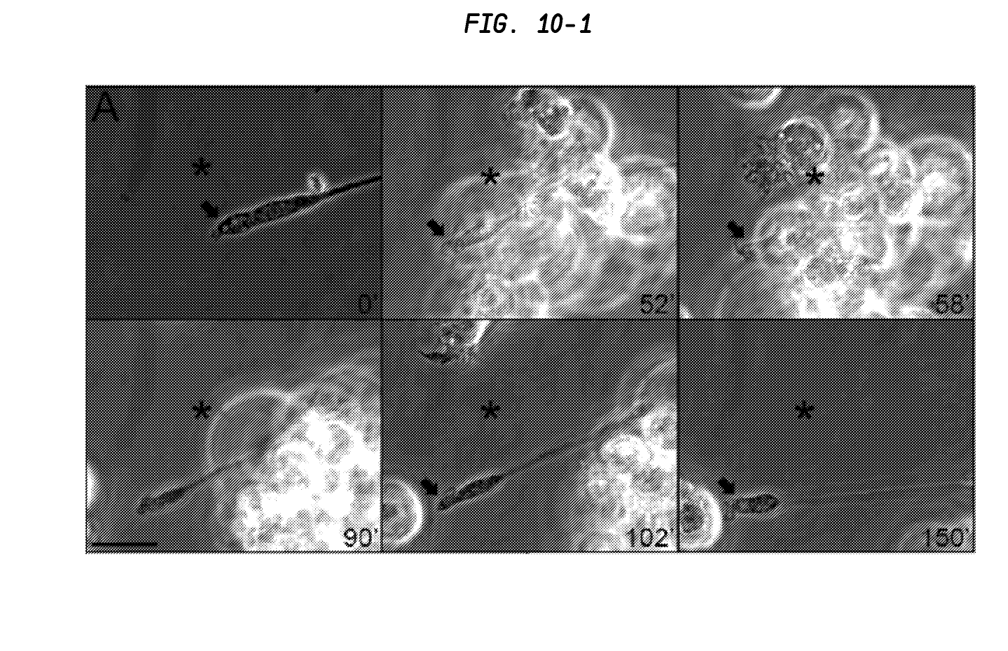

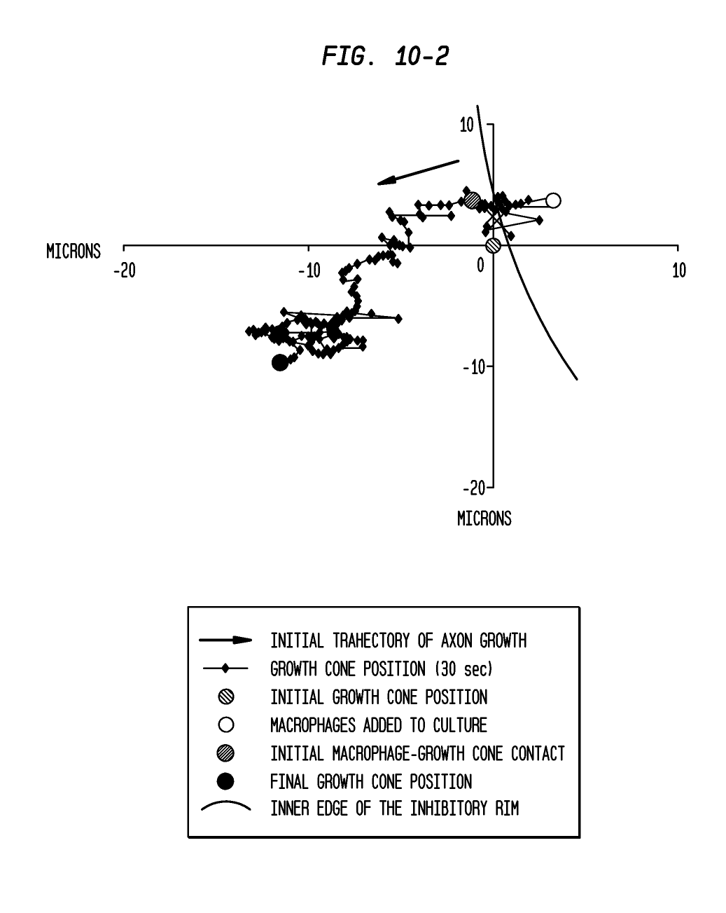

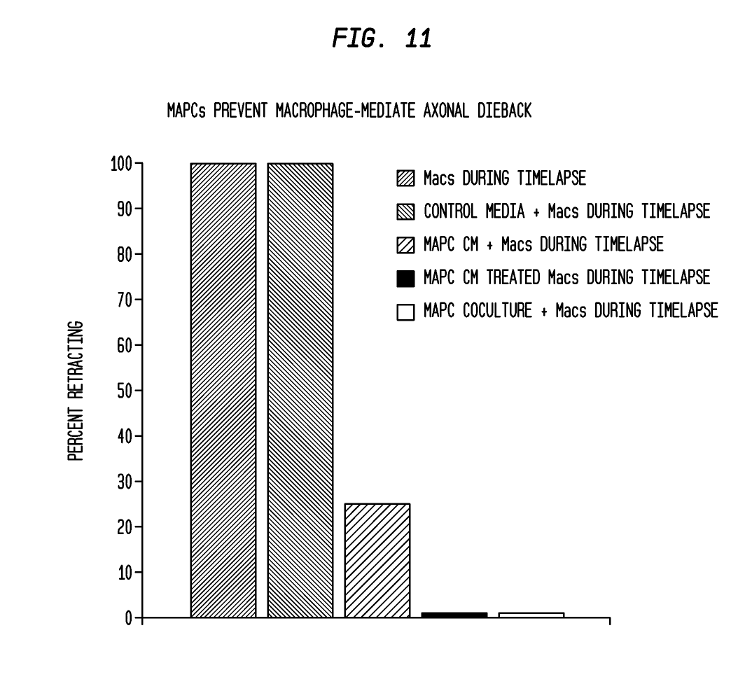

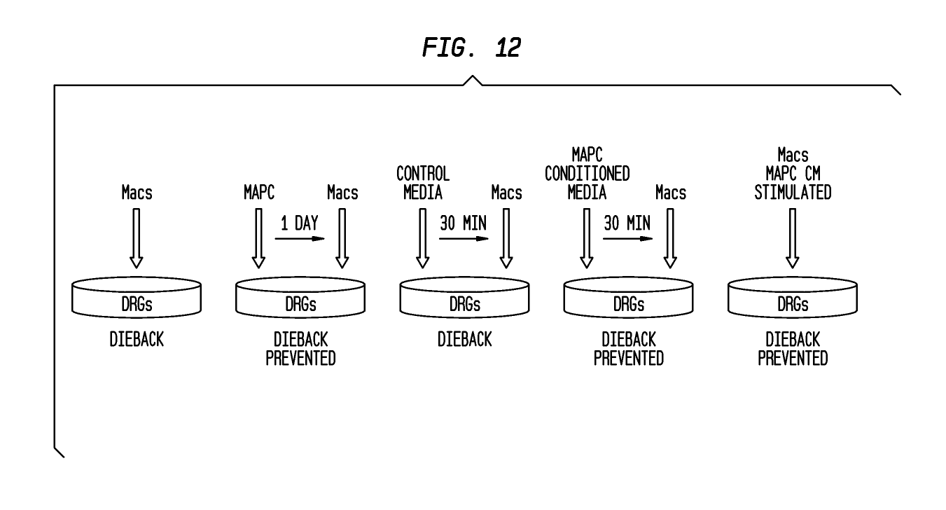

Direct addition of MAPC-conditioned media to the timelapse dish resulted in a change in growth cone morphology, from a dystrophic, stalled state, to a motile, flattened state. Macrophages still contacted these axons, but contacts were generally transient and generally did not result in axonal retraction. Control MAPC media did not prevent axonal retraction. Macrophages pretreated with MAPC-conditioned media also contacted axons on the spot, but did not cause retraction (FIGS. 9-12). It is possible that MAPCs act on macrophages to alter their receptor expression, response to injured cells, or secretion of MMP-9.

### DETAILED DESCRIPTION OF THE INVENTION

### Definitions

“A” or “an” means herein one or more than one; at least one. Where the plural form is used herein, it generally includes the singular.

As used herein, the terms “adhere(s), adherence, adhesion”, and the like, refer to an association of sufficient duration so as to induce axonal retraction. As described further herein, it is understood that physical contact may occur between macrophages (or other cells) and dystrophic axons that is transient and does not result in axonal retraction. Within the context of the invention, the adherence that is reduced or prevented by the reagents of the invention is that which occurs for sufficient duration so as to induce axonal retraction. Thus, the invention does not exclude reagents that allow physical contact between dystrophic axons and ED-1⁺ cells. The invention thus encompasses reagents that allow contact (such as transient physical contact) but do not allow adherence for time sufficient to result in axonal dieback.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of comprising (see above).

“Conditioned cell culture medium” is a term well-known in the art and refers to medium in which cells have been grown. Herein this means that the cells are grown for a sufficient time to secrete the factors that are effective to reduce the adhesion of activated macrophages to dystrophic neurons and/or promote neurite outgrowth/axon regeneration.

Conditioned cell culture medium refers to medium in which cells have been cultured so as to secrete factors into the medium. For the purposes of the present invention, cells can be grown through a sufficient number of cell divisions so as to produce effective amounts of such factors so that the medium reduces the adhesion of macrophages to dystrophic neurons and hence reduces axonal retraction and/or promote neurite outgrowth/axon regeneration. Cells are removed from the medium by any of the known methods in the art, including, but not limited to, centrifugation, filtration, immunodepletion (e.g., via tagged antibodies and magnetic columns), and FACS sorting.

“Dieback” is a term of art used to refer to axonal retraction that occurs as a result of trauma to the axon. The axonal retraction, within the context of the invention, refers to that which occurs as a result of sufficient adherence of ED-1⁺ cells and, particularly, macrophages and microglia. Such macrophages and microglia (i.e., ED-1⁺ cells) are not in the resting or inactive state. They are activated. The term “activated” refers to a state of these cells that allows them to adhere to a dystrophic axon so as to result in axonal retraction. Examples of conditions resulting in activation in vitro are described further in this application. It is to be understood, however, that such activation is not limited to the specific conditions disclosed herein.

Although the invention is often specifically directed to (and exemplified by) activated macrophages, these are a class of ED-1⁺ cells and the invention pertains to other such cells. One example is activated microglia.

“Effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

“EC cells” were discovered from analysis of a type of cancer called a teratocarcinoma. In 1964, researchers noted that a single cell in teratocarcinomas could be isolated and remain undifferentiated in culture. This type of stem cell became known as an embryonic carcinoma cell (EC cell).

“Embryonic Stem Cells (ESC)” are well known in the art and have been prepared from many different mammalian species for many years. Embryonic stem cells are stem cells derived from the inner cell mass of an early stage embryo known as a blastocyst. They are able to differentiate into all derivatives of the three primary germ layers: ectoderm, endoderm, and mesoderm. These include each of the more than 220 cell types in the adult body. The ES cells can become any tissue in the body, excluding placenta. Only the morula's cells are totipotent, able to become all tissues and a placenta.

Use of the term “includes” is not intended to be limiting. For example, stating that the antibody inhibitor “includes” fragments and variants does not mean that other forms of the antibody inhibitor are excluded.

“Induced pluripotent stem cells (IPSC or IPS cells)” are somatic cells that have been reprogrammed. for example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them. It is also possible that such cells can be created by specific culture conditions (exposure to specific agents).

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only stem cells. Rather, the term “isolated” indicates that the cells are removed from their natural tissue environment and are present at a higher concentration as compared to the normal tissue environment. Accordingly, an “isolated” cell population may further include cell types in addition to stem cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (e.g., bone marrow, peripheral blood, adipose tissue, etc.).

“MAPC” is an acronym for “multipotent adult progenitor cell.” It refers to a non-embryonic stem cell. The term “adult” in MAPC is non-restrictive. It refers to a non-embryonic somatic cell. Like embryonic stem cells, the MAPC can give rise to cell lineages of more than one germ layer. It may give rise to cell types of all three germ layers (i.e., endoderm, mesoderm and ectoderm) upon differentiation. Like embryonic stem cells, human MAPCs express telomerase, Oct 3/4 (i.e., Oct 3A), rex-1, rox-1 and sox-2, and may express SSEA-4. (See also Jiang, Y. et al., Nature, 418:41 (2002); Exp Hematol, 30:896 (2002)). The telomeres are extended in MAPCs and they are karyotypically normal. Because MAPCs injected into a mammal can migrate to and assimilate within multiple organs, MAPCs are self-renewing stem cells. “Multipotent”, with respect to MAPC, refers to the ability to give rise to cell lineages of more than more than one primitive germ layer (i.e., endoderm, mesoderm and ectoderm) upon differentiation, such as all three.

“Neurite outgrowth” refers to the property of neurons at the site of the injury not only to cease to retract but to grow and extend.

“Pharmaceutically acceptable carrier” is any pharmaceutically acceptable medium for the cells used in the present invention. Such a medium may retain isotonicity, cell metabolism, pH, and the like. It is compatible with administration to a subject in vivo, and can be used, therefore, for cell delivery and treatment.

“Primordial embryonic germ cells” (PG or EG cells) can be cultured and stimulated to produce many less differentiated cell types.

“Progenitor cells” are cells produced during differentiation of a stem cell that have some, but not all, of the characteristics of their terminally-differentiated progeny. Defined progenitor cells, such as “cardiac progenitor cells,” are committed to a lineage, but not to a specific or terminally differentiated cell type. The term “progenitor” as used in the acronym “MAPC” does not limit these cells to a particular lineage.

The term “reduce” as used herein means to prevent as well as decrease. In the context of treatment, to “reduce” is to both prevent or ameliorate one or more clinical symptoms. A clinical symptom is one (or more) that has or will have, if left untreated, a negative impact on the quality of life (health) of the subject.

The term “retraction” refers to the receding of the axon away from the site of injury, such as from where the glial scar forms. Here, the end of regenerating axons stop extending and become dystrophic. These dystrophic ends then can recede further from the glial scar and the site of injury.

“Self-renewal” refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has dedifferentiated, for example, by nuclear transfer, by fusions with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation. Stem cells also may be derived from abnormal tissue, such as a teratocarcinoma and some other sources such as embryoid bodies (although these can be considered embryonic stem cells in that they are derived from embryonic tissue, although not directly from the inner cell mass). Stem cells may also be produced by introducing genes associated with stem cell function into a non-stem cell, such as an induced pluripotent stem cell.

“Subject” means a vertebrate, such as a mammal, such as a human. Mammals include, but are not limited to, humans, dogs, cats, horses, cows, and pigs.

The term “therapeutically effective amount” refers to the amount determined to produce any therapeutic response in a mammal. For example, effective amounts of the therapeutic cells or cell-associated agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms that occur from the adherence of activated macrophages to dystrophic axons. Treating also refers to the beneficial clinical effect of axon regeneration.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

### Stem Cells

The present invention can be practiced, preferably, using stem cells of vertebrate species, such as humans, non-human primates, domestic animals, livestock, and other non-human mammals. These include, but are not limited to, those cells described below.

Embryonic Stem Cells

The most well studied stem cell is the embryonic stem cell (ESC) as it has unlimited self-renewal and multipotent differentiation potential. These cells are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

A number of transcription factors and exogenous cytokines have been identified that influence the potency status of embryonic stem cells in vivo. The first transcription factor to be described that is involved in stem cell pluripotency is Oct4. Oct4 belongs to the POU (Pit-Oct-Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct3/4 is to repress differentiation inducing genes (i.e., FoxaD3, hCG) and to activate genes promoting pluripotency (FGF4, Utf1, Rex1). Sox2, a member of the high mobility group (HMG) box transcription factors, cooperates with Oct4 to activate transcription of genes expressed in the inner cell mass. It is essential that Oct3/4 expression in embryonic stem cells is maintained between certain levels. Overexpression or downregulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage. Sall4, a mammalian Spalt transcription factor, is an upstream regulator of Oct4, and is therefore important to maintain appropriate levels of Oct4 during early phases of embryology. When Sall4 levels fall below a certain threshold, trophectodermal cells will expand ectopically into the inner cell mass. Another transcription factor required for pluripotency is Nanog, named after a celtic tribe “Tir Nan Og”: the land of the ever young. In vivo, Nanog is expressed from the stage of the compacted morula, is subsequently defined to the inner cell mass and is downregulated by the implantation stage. Downregulation of Nanog may be important to avoid an uncontrolled expansion of pluripotent cells and to allow multilineage differentiation during gastrulation. Nanog null embryos, isolated at day 5.5, consist of a disorganized blastocyst, mainly containing extraembryonic endoderm and no discernable epiblast.

Non-Embryonic Stem Cells

Stem cells have been identified in most tissues. Perhaps the best characterized is the hematopoietic stem cell (HSC). HSCs are mesoderm-derived cells that can be purified using cell surface markers and functional characteristics. They have been isolated from bone marrow, peripheral blood, cord blood, fetal liver, and yolk sac. They initiate hematopoiesis and generate multiple hematopoietic lineages. When transplanted into lethally-irradiated animals, they can repopulate the erythroid neutrophil-macrophage, megakaryocyte, and lymphoid hematopoietic cell pool. They can also be induced to undergo some self-renewal cell division. See, for example, U.S. Pat. Nos. 5,635,387; 5,460,964; 5,677,136; 5,750,397; 5,681,599; and 5,716,827. U.S. Pat. No. 5,192,553 reports methods for isolating human neonatal or fetal hematopoietic stem or progenitor cells. U.S. Pat. No. 5,716,827 reports human hematopoietic cells that are Thy-1⁺ progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells. Accordingly, HSCs and methods for isolating and expanding them are well-known in the art.

Another stem cell that is well-known in the art is the neural stem cell (NSC). These cells can proliferate in vivo and continuously regenerate at least some neuronal cells. When cultured ex vivo, neural stem cells can be induced to proliferate as well as differentiate into different types of neurons and glial cells. When transplanted into the brain, neural stem cells can engraft and generate neural and glial cells. See, for example, Gage F. H., Science, 287:1433-1438 (2000), Svendsen S, N. et al, Brain Pathology, 9:499-513 (1999), and Okabe S. et al., Mech Development, 59:89-102 (1996). U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,564,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain. Accordingly, neural stem cells and methods for making and expanding them are well-known in the art.

Another stem cell that has been studied extensively in the art is the mesenchymal stem cell (MSC). MSCs are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

Another example of an adult stem cell is adipose-derived adult stem cells (ADSCs) which have been isolated from fat, typically by liposuction followed by release of the ADSCs using collagenase. ADSCs are similar in many ways to MSCs derived from bone marrow, except that it is possible to isolate many more cells from fat. These cells have been reported to differentiate into bone, fat, muscle, cartilage, and neurons. A method of isolation has been described in U.S. 2005/0153442.

Other stem cells that are known in the art include gastrointestinal stem cells, epidermal stem cells, and hepatic stem cells, which have also been termed “oval cells” (Potten, C., et al., Trans R Soc Lond B Biol Sci, 353:821-830 (1998), Watt, F., Trans R Soc Lond B Biol Sci, 353:831 (1997); Alison et al., Hepatology, 29:678-683 (1998).

Other non-embryonic cells reported to be capable of differentiating into cell types of more than one embryonic germ layer include, but are not limited to, cells from umbilical cord blood (see U.S. Publication No. 2002/0164794), placenta (see U.S. Publication No. 2003/0181269, umbilical cord matrix (Mitchell, K. E. et al., Stem Cells, 21:50-60 (2003)), small embryonic-like stem cells (Kucia, M. et al., J Physiol Pharmacol, 57 Suppl 5:5-18 (2006)), amniotic fluid stem cells (Atala, A., J Tissue Regen Med, 1:83-96 (2007)), skin-derived precursors (Toma et al., Nat Cell Biol, 3:778-784 (2001)), and bone marrow (see U.S. Publication Nos. 2003/0059414 and 2006/0147246), each of which is incorporated by reference for teaching these cells.

Strategies of Reprogramming Somatic Cells

Several different strategies such as nuclear transplantation, cellular fusion, and culture induced reprogramming have been employed to induce the conversion of differentiated cells into an embryonic state. Nuclear transfer involves the injection of a somatic nucleus into an enucleated oocyte, which, upon transfer into a surrogate mother, can give rise to a clone (“reproductive cloning”), or, upon explantation in culture, can give rise to genetically matched embryonic stem (ES) cells (“somatic cell nuclear transfer,” SCNT). Cell fusion of somatic cells with ES cells results in the generation of hybrids that show all features of pluripotent ES cells. Explantation of somatic cells in culture selects for immortal cell lines that may be pluripotent or multipotent. At present, spermatogonial stem cells are the only source of pluripotent cells that can be derived from postnatal animals. Transduction of somatic cells with defined factors can initiate reprogramming to a pluripotent state. These experimental approaches have been extensively reviewed (Hochedlinger and Jaenisch, Nature, 441:1061-1067 (2006) and Yamanaka, S., Cell Stem Cell, 1:39-49 (2007)).

Nuclear Transfer

Nuclear transplantation (NT), also referred to as somatic cell nuclear transfer (SCNT), denotes the introduction of a nucleus from a donor somatic cell into an enucleated ogocyte to generate a cloned animal such as Dolly the sheep (Wilmut et al., Nature, 385:810-813 (1997). The generation of live animals by NT demonstrated that the epigenetic state of somatic cells, including that of terminally differentiated cells, while stable, is not irreversible fixed but can be reprogrammed to an embryonic state that is capable of directing development of a new organism. In addition to providing an exciting experimental approach for elucidating the basic epigenetic mechanisms involved in embryonic development and disease, nuclear cloning technology is of potential interest for patient-specific transplantation medicine.

Fusion of Somatic Cells and Embryonic Stem Cells

Epigenetic reprogramming of somatic nuclei to an undifferentiated state has been demonstrated in murine hybrids produced by fusion of embryonic cells with somatic cells. Hybrids between various somatic cells and embryonic carcinoma cells (Solter, D., Nat Rev Genet, 7:319-327 (2006), embryonic germ (EG), or ES cells (Zwaka and Thomson, Development, 132:227-233 (2005)) share many features with the parental embryonic cells, indicating that the pluripotent phenotype is dominant in such fusion products. As with mouse (Tada et al., Curr Biol, 11:1553-1558 (2001)), human ES cells have the potential to reprogram somatic nuclei after fusion (Cowan et al., Science, 309:1369-1373 (2005)); Yu et al., Science, 318:1917-1920 (2006)). Activation of silent pluripotency markers such as Oct4 or reactivation of the inactive somatic X chromosome provided molecular evidence for reprogramming of the somatic genome in the hybrid cells. It has been suggested that DNA replication is essential for the activation of pluripotency markers, which is first observed 2 days after fusion (Do and Scholer, Stem Cells, 22:941-949 (2004)), and that forced overexpression of Nanog in ES cells promotes pluripotency when fused with neural stem cells (Silva et al., Nature, 441:997-1001 (2006)).

### Culture-Induced Reprogramming

Pluripotent cells have been derived from embryonic sources such as blastomeres and the inner cell mass (ICM) of the blastocyst (ES cells), the epiblast (EpiSC cells), primordial germ cells (EG cells), and postnatal spermatogonial stem cells (“maGSCsm” “ES-like” cells). The following pluripotent cells, along with their donor cell/tissue is as follows: parthogenetic ES cells are derived from murine oocytes (Narasimha et al., Curr Biol, 7:881-884 (1997)); embryonic stem cells have been derived from blastomeres (Wakayama et al., Stem Cells, 25:986-993 (2007)); inner cell mass cells (source not applicable) (Eggan et al., Nature, 428:44-49 (2004)); embryonic germ and embryonal carcinoma cells have been derived from primordial germ cells (Matsui et al., Cell, 70:841-847 (1992)); GMCS, maSSC, and MASC have been derived from spermatogonial stem cells (Guan et al., Nature, 440:1199-1203 (2006); Kanatsu-Shinohara et al., Cell, 119:1001-1012 (2004); and Seandel et al., Nature, 449:346-350 (2007)); EpiSC cells are derived from epiblasts (Brons et al., Nature, 448:191-195 (2007); Tesar et al., Nature, 448:196-199 (2007)); parthogenetic ES cells have been derived from human oocytes (Cibelli et al., Science, 295L819 (2002); Revazova et al., Cloning Stem Cells, 9:432-449 (2007)); human ES cells have been derived from human blastocysts (Thomson et al., Science, 282:1145-1147 (1998)); MAPC have been derived from bone marrow (Jiang et al., Nature, 418:41-49 (2002); Phinney and Prockop, Stem Cells, 25:2896-2902 (2007)); cord blood cells (derived from cord blood) (van de Ven et al., Exp Hematol, 35:1753-1765 (2007)); neurosphere derived cells derived from neural cell (Clarke et al., Science, 288:1660-1663 (2000)). Donor cells from the germ cell lineage such as PGCs or spermatogonia) stem cells are known to be unipotent in vivo, but it has been shown that pluripotent ES-like cells (Kanatsu-Shinohara et al., Cell, 119:1001-1012 (2004) or maGSCs (Guan et al., Nature, 440:1199-1203 (2006), can be isolated after prolonged in vitro culture. While most of these pluripotent cell types were capable of in vitro differentiation and teratoma formation, only ES, EG, EC, and the spermatogonia) stem cell-derived maGCSs or ES-like cells were pluripotent by more stringent criteria, as they were able to form postnatal chimeras and contribute to the germline. Recently, multipotent adult spermatogonia) stem cells (MASCs) were derived from testicular spermatogonia) stem cells of adult mice, and these cells had an expression profile different from that of ES cells (Seandel et al., Nature, 449:346-350 (2007)) but similar to EpiSC cells, which were derived from the epiblast of postimplantation mouse embryos (Brons et al., Nature, 448:191-195 (2007); Tesar et al., Nature, 448:196-199 (2007)).

Reprogramming by Defined Transcription Factors

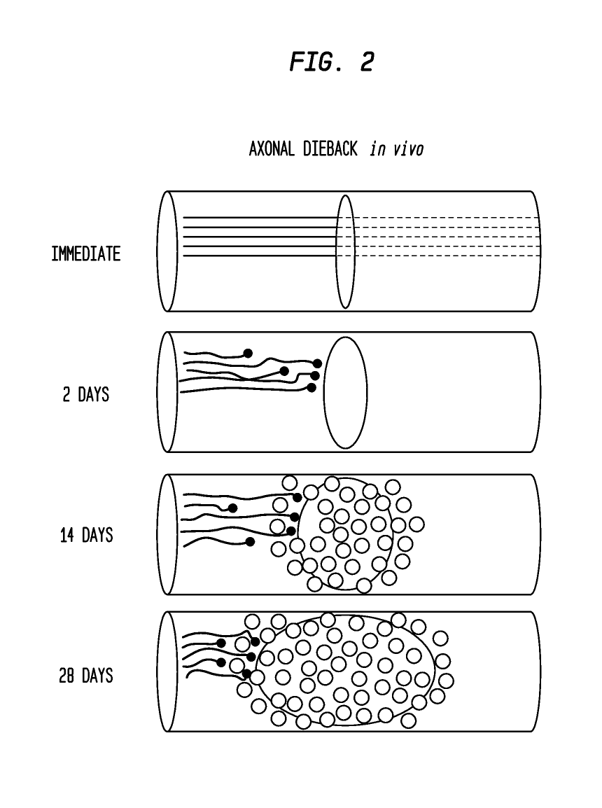

Takahashi and Yamanaka have reported reprogramming somatic cells back to an ES-like state (Takahashi and Yamanaka, Cell, 126:663-676 (2006)). They successfully reprogrammed mouse embryonic fibroblasts (MEFs) and adult fibroblasts to pluripotent ES-like cells after viral-mediated transduction of the four transcription factors Oct4, Sox2, c-myc, and Klf4 followed by selection for activation of the Oct4 target gene Fbx15 (FIG. 2A). Cells that had activated Fbx15 were coined iPS (induced pluripotent stem) cells and were shown to be pluripotent by their ability to form teratomas, although the were unable to generate live chimeras. This pluripotent state was dependent on the continuous viral expression of the transduced Oct4 and Sox2 genes, whereas the endogenous Oct4 and Nanog genes were either not expressed or were expressed at a lower level than in ES cells, and their respective promoters were found to be largely methylated. This is consistent with the conclusion that the Fbx15-iPS cells did not correspond to ES cells but may have represented an incomplete state of reprogramming. While genetic experiments had established that Oct4 and Sox2 are essential for pluripotency (Chambers and Smith, Oncogene, 23:7150-7160 (2004); Ivanona et al., Nature, 442:5330538 (2006); Masui et al., Nat Cell Biol, 9:625-635 (2007)), the role of the two oncogenes c-myc and Klf4 in reprogramming is less clear. Some of these oncogenes may, in fact, be dispensable for reprogramming, as both mouse and human iPS cells have been obtained in the absence of c-myc transduction, although with low efficiency (Nakagawa et al., Nat Biotechnol, 26:191-106 (2008); Werning et al., Nature, 448:318-324 (2008); Yu et al., Science, 318: 1917-1920 (2007)).

### MAPC

MAPC is an acronym for “multipotent adult progenitor cell” (non-ES, non-EG, non-germ). MAPC have the capacity to differentiate into cell types of at least two, such as, all three, primitive germ layers (ectoderm, mesoderm, and endoderm). Genes found in ES cells were also found in MAPC (e.g., telomerase, Oct 3/4, rex-1, rox-1, sox-2). Oct 3/4 (Oct 3A in humans) appears to be specific for ES and germ cells. MAPC represents a more primitive progenitor cell population than MSC and demonstrates differentiation capability encompassing the epithelial, endothelial, neural, myogenic, hematopoietic, osteogenic, hepatogenic, chondrogenic and adipogenic lineages (Verfallie, C. M., Trends Cell Biol 12:502-8 (2002), Jahagirdar, B. N., et al., Exp Hematol, 29:543-56 (2001); Reyes, M. and C. M. Verfallie, Ann N Y Acad Sci, 938:231-233 (2001); Jiang, Y. et al., Exp Hematol, 30896-904 (2002); and (Jiang, Y. et al., Nature, 418:41-9. (2002)).

Human MAPCs are described in U.S. Pat. No. 7,015,037 and U.S. application Ser. No. 10/467,963. MAPCs have been identified in other mammals. Murine MAPCs, for example, are also described in U.S. Pat. No. 7,015,037 and U.S. application Ser. No. 10/467,963. Rat MAPCs are also described in U.S. application Ser. No. 10/467,963.

These references are incorporated by reference for describing MAPCs isolated by Catherine Verfallie.

### Isolation and Growth of MAPCs

Methods of MAPC isolation are known in the art. See, for example, U.S. Pat. No. 7,015,037 and U.S. application Ser. No. 10/467,963, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs from Human Bone Marrow as Described in U.S. Pat. No. 7,015,037

MAPCs do not express the common leukocyte antigen CD45 or erythroblast specific glycophorin-A (Gly-A). The mixed population of cells was subjected to a Ficoll Hypaque separation. The cells were then subjected to negative selection using anti-CD45 and anti-Gly-A antibodies, depleting the population of CD45⁺ and Gly-A⁺ cells, and the remaining approximately 0.1% of marrow mononuclear cells were then recovered. Cells could also be plated in fibronectin-coated wells and cultured as described below for 2-4 weeks to deplete the cells of CD45⁺ and Gly-A⁺ cells. In cultures of adherent bone marrow cells, many adherent stromal cells undergo replicative senescence around cell doubling 30 and a more homogenous population of cells continues to expand and maintains long telomeres.

Alternatively, positive selection could be used to isolate cells via a combination of cell-specific markers. Both positive and negative selection techniques are available to those of skill in the art, and numerous monoclonal and polyclonal antibodies suitable for negative selection purposes are also available in the art (see, for example, Leukocyte Typing V, Schlossman, et al., Eds. (1995) Oxford University Press) and are commercially available from a number of sources.

Techniques for mammalian cell separation from a mixture of cell populations have also been described by Schwartz, et al., in U.S. Pat. No. 5,759,793 (magnetic separation), Basch et al., 1983 (immunoaffinity chromatography), and Wysocki and Sato, 1978 (fluorescence-activated cell sorting).

Culturing MAPCs as Described in U.S. Pat. No. 7,015,037

MAPCs isolated as described herein can be cultured using methods disclosed herein and in U.S. Pat. No. 7,015,037, which is incorporated by reference for these methods.

### Additional Culture Methods

In additional experiments the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 1-5% and, especially, 3-5%, can be used at any time during the isolation, growth and differentiation of MAPCs in culture.

Cells may be cultured under various serum concentrations, e.g., about 2-20%. Fetal bovine serum may be used. Higher serum may be used in combination with lower oxygen tensions, for example, about 15-20%. Cells need not be selected prior to adherence to culture dishes. For example, after a ficoll gradient, cells can be directly plated, e.g., 250,000-500,000/cm². Adherent colonies can be picked, possibly pooled, and expanded.

In one embodiment, used in the experimental procedures in the Examples, high serum (around 15-20%) and low oxygen (around 3-5%) conditions were used for the cell culture. Specifically, adherent cells from colonies were plated and passaged at densities of about 1700-2300 cells/cm² in 18% serum and 3% oxygen (with PDGF and EGF).

In an embodiment specific for MAPCs, supplements are cellular factors or components that allow MAPCs to retain the ability to differentiate into all three lineages. This may be indicated by the expression of specific markers of the undifferentiated state. MAPCs, for example, constitutively express Oct 3/4 (Oct 3A) and maintain high levels of telomerase.

### Cell Culture

In general, cells useful for the invention can be maintained and expanded in culture medium that is available to and well-known in the art. Such media include, but are not limited to Dulbecco's Modified Eagle's Medium® (DMEM), DMEM F12 Medium®, Eagle's Minimum Essential Medium®, F-12K Medium®, Iscove's Modified Dulbecco's Medium® and RPMI-1640 Medium®. Many media are also available as a low-glucose formulations, with or without sodium pyruvate.

Also contemplated is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements, and bovine embryonic fluid. It is understood that sera can be heat-inactivated at 55-65° C. if deemed necessary to inactivate components of the complement cascade.

Additional supplements can also be used advantageously to supply the cells with the necessary trace elements for optimal growth and expansion. Such supplements include insulin, transferrin, sodium selenium and combinations thereof. These components can be included in a salt solution such as, but not limited to Hanks' Balanced Salt Solution® (HBSS), Earle's Salt Solution®, antioxidant supplements, MCDB-201® supplements, phosphate buffered saline (PBS), ascorbic acid and ascorbic acid-2-phosphate, as well as additional amino acids. Many cell culture media already contain amino acids, however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Hormones can also be advantageously used in cell culture and include, but are not limited to D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine.

Lipids and lipid carriers can also be used to supplement cell culture media, depending on the type of cell and the fate of the differentiated cell. Such lipids and carriers can include, but are not limited to cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

Also contemplated is the use of feeder cell layers. Feeder cells are used to support the growth of fastidious cultured cells, particularly ES cells. Feeder cells are normal cells that have been inactivated by γ-irradiation. In culture, the feeder layer serves as a basal layer for other cells and supplies cellular factors without further growth or division of their own (Lim, J. W. and Bodnar, A., 2002). Examples of feeder layer cells are typically human diploid lung cells, mouse embryonic fibroblasts, Swiss mouse embryonic fibroblasts, but can be any post-mitotic cell that is capable of supplying cellular components and factors that are advantageous in allowing optimal growth, viability, and expansion of stem cells. In many cases, feeder cell layers are not necessary to keep the ES cells in an undifferentiated, proliferative state, as leukemia inhibitory factor (LIF) has anti-differentiation properties. Therefore, supplementation with LIF could be used to maintain MAPC in some species in an undifferentiated state.

Cells may be cultured in low-serum or serum-free culture medium. Serum-free medium used to culture MAPCs is described in U.S. Pat. No. 7,015,037. Many cells have been grown in serum-free or low-serum medium. In this case, the medium is supplemented with one or more growth factors. Commonly-used growth factors include but are not limited to bone morphogenic protein, basis fibroblast growth factor, platelet-derived growth factor, and epidermal growth factor. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference for teaching growing cells in serum-free medium.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. See, for example, Ohashi et al., Nature Medicine, 13:880-885 (2007); Matsumoto et al., J Bioscience and Bioengineering, 105:350-354 (2008); Kirouac et al., Cell Stem Cell, 3:369-381 (2008); Chua et al., Biomaterials, 26:2537-2547 (2005); Drobinskaya et al., Stem Cells, 26:2245-2256 (2008); Dvir-Ginzberg et al., FASEB J, 22:1440-1449 (2008); Turner et al., J Biomed Mater Res Part B: Appl Biomater, 82B:156-168 (2007); and Miyazawa et al., Journal of Gastroenterology and Hepatology, 22:1959-1964 (2007)).

Cells may also be grown in “3D” (aggregated) cultures. An example is U.S. Provisional Patent Application No. 61/022,121, filed Jan. 18, 2008.

Once established in culture, cells can be used fresh or frozen and stored as frozen stocks, using, for example, DMEM with 40% FCS and 10% DMSO. Other methods for preparing frozen stocks for cultured cells are also available to those of skill in the art.

### Pharmaceutical Formulations

In certain embodiments, the purified cell populations are present within a composition adapted for and suitable for delivery, i.e., physiologically compatible. Accordingly, compositions of the stem cell populations will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

In other embodiments, the purified cell populations are present within a composition adapted for or suitable for freezing or storage.

In many embodiments the purity of the cells (or conditioned medium) for administration to a subject is about 100%. In other embodiments it is 95% to 100%. In some embodiments it is 85% to 95%. Particularly in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

The numbers of cells in a given volume can be determined by well known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The choice of formulation for administering the cells for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Various embodiments of the invention comprise measures to increase cell survival and/or to overcome problems posed by barriers to seeding and/or growth.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose and the like. Organic polymer base materials, such as polyethylene glycol and hyaluronic acid as well as non-fibrillar collagen, preferably succinylated collagen, can also act as lubricants. Such lubricants are generally used to improve the injectability, intrudability and dispersion of the injected biomaterial at the site of injection and to decrease the amount of spiking by modifying the viscosity of the compositions. This final formulation is by definition the cells in a pharmaceutically acceptable carrier.

The cells are subsequently placed in a syringe or other injection apparatus for precise placement at the site of the tissue defect. The term “injectable” means the formulation can be dispensed from syringes having a gauge as low as 25 under normal conditions under normal pressure without substantial spiking. Spiking can cause the composition to ooze from the syringe rather than be injected into the tissue. For this precise placement, needles as fine as 27 gauge (200μ I.D.) or even 30 gauge (150μ I.D.) are desirable. The maximum particle size that can be extruded through such needles will be a complex function of at least the following: particle maximum dimension, particle aspect ratio (length:width), particle rigidity, surface roughness of particles and related factors affecting particle:particle adhesion, the viscoelastic properties of the suspending fluid, and the rate of flow through the needle. Rigid spherical beads suspended in a Newtonian fluid represent the simplest case, while fibrous or branched particles in a viscoelastic fluid are likely to be more complex.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or stabilizer can be employed to increase the life of cell/medium compositions. If such preservatives are included, it is well within the purview of the skilled artisan to select compositions that will not affect the viability or efficacy of the cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells/medium utilized in practicing the present invention in the required amount of the appropriate solvent with various amounts of the other ingredients, as desired.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments cells are encapsulated for administration, particularly where encapsulation enhances the effectiveness of the therapy, or provides advantages in handling and/or shelf life. Encapsulation in some embodiments where it increases the efficacy of cell mediated immunosuppression may, as a result, also reduce the need for immunosuppressive drug therapy.

Also, encapsulation in some embodiments provides a barrier to a subject's immune system that may further reduce a subject's immune response to the cells (which generally are not immunogenic or are only weakly immunogenic in allogeneic transplants), thereby reducing any graft rejection or inflammation that might occur upon administration of the cells.

Cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation of cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of cells.

Certain embodiments incorporate cells into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the invention, cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

### Dosing

Compositions can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of cells/medium appropriate to be used in accordance with various embodiments of the invention will depend on numerous factors. It may vary considerably for different circumstances. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration. Finally, the determination of optimal dosing necessarily will provide an effective dose that is neither below the threshold of maximal beneficial effect nor above the threshold where the deleterious effects associated with the dose outweighs the advantages of the increased dose.

The optimal dose of cells for some embodiments will be in the range of doses used for autologous, mononuclear bone marrow transplantation. For fairly pure preparations of cells, optimal doses in various embodiments will range from 10⁴ to 10⁸ cells/kg of recipient mass per administration. In some embodiments the optimal dose per administration will be between 10⁵ to 10⁷ cells/kg. In many embodiments the optimal dose per administration will be 5×10⁵ to 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, cells/medium may be administered in an initial dose, and thereafter maintained by further administration. Cells/medium may be administered by one method initially, and thereafter administered by the same method or one or more different methods. The levels can be maintained by the ongoing administration of the cells/medium. Various embodiments administer the cells/medium either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regimens can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer the cells/medium.

In some embodiments cells/medium are administered to a subject in one dose. In others cells/medium are administered to a subject in a series of two or more doses in succession. In some other embodiments wherein cells/medium are administered in a single dose, in two doses, and/or more than two doses, the doses may be the same or different, and they are administered with equal or with unequal intervals between them.

Cells/medium may be administered in many frequencies over a wide range of times. In some embodiments, they are administered over a period of less than one day. In other embodiment they are administered over two, three, four, five, or six days. In some embodiments they are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

For example, for spinal cord injury it is explained in this document that there may be two phases. In the animal models, in the first phase, macrophages do not infiltrate the lesion and this lasts for about 24 hours. In the second phase, however, macrophages do infiltrate the lesion and this sequence of events may be taken into consideration when assessing treatment regimens. In one embodiment, cells are administered even during the first phase, such as immediately after injury or as close to the injury as possible in anticipation of the infiltration of macrophages or other cells that would interact with dystrophic axons. Treatment may then be continued to coincide with initial and further infiltration of macrophages and may be preventatively continued or possibly discontinued when it is determined that macrophages or the other relevant cells are no longer infiltrating the injury.

### EXAMPLES

### Example I

“Glial Scar Model” Aggrecan-laminin opposing spot gradients (Tom et al., 2004; Steinmetz et al., 2005). These references are incorporated by reference for teaching the glial scar model. This model provides an assay for the effectiveness of cells, proteins, medium, etc., in reducing adhesion/retraction in vitro.

PGs can induce the so-called dystrophic state in axons if the inhibitory matrix is presented in a spatial organization that more closely resembles that which develops after lesions in vivo. To do this, spots of a solution of the PG aggrecan and the growth-promoting molecule laminin were placed on nitrocellulose coverslips and air dried.

A consistent artifact of drying produced a crude gradient in which the rim of the spot contained an increasingly higher concentration of aggrecan than in the center. The very outermost part of the rim contained a lower concentration of laminin than any more central region. The optimal ECM concentrations (0.7 mg/ml aggrecan and 5 μg/ml laminin) resulted in good cell attachment. Thus, the high aggrecan-low laminin outer rim appeared to be a particularly harsh terrain for regenerating neurites. None entered inward into the spot from the laminin surround by crossing its sharp outer interface. Fibers growing centripetally from within the center of the spot were able to enter the inner portion of the rim but could grow no farther. Once within the gradient, axons appeared trapped. Club-like, dystrophic endballs formed at the ends of neurites within the gradient. To observe the behavior of the “dystrophic endings” time-lapse microscopy was used. Dystrophic growth cones often managed to advance short distances, but inevitably, the struggling growth cone would round up into a more compact ball and retract, only to start moving again.

### Example II

### Axonal Retraction and Macrophages

### Summary

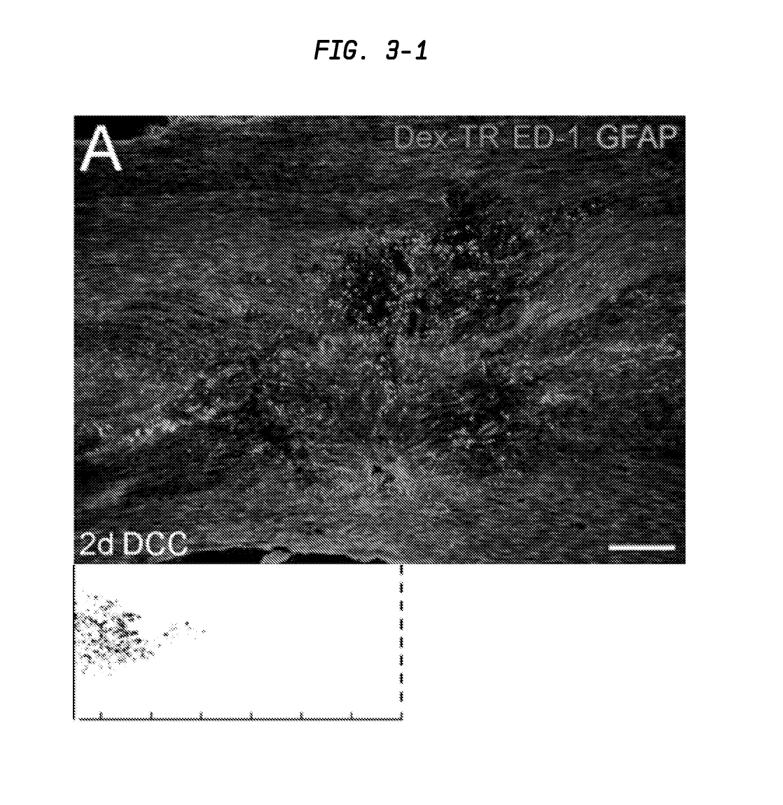

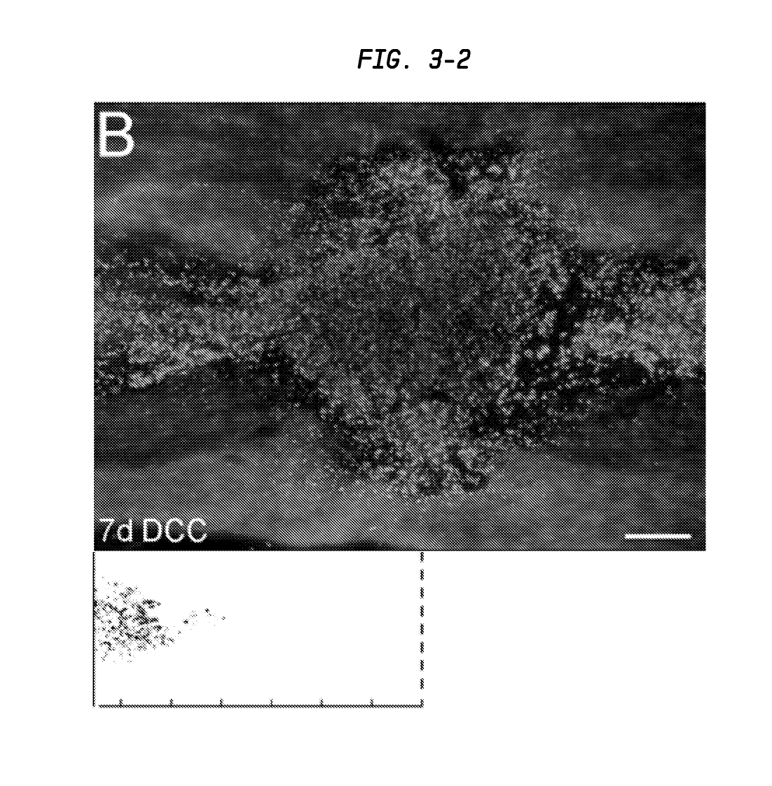

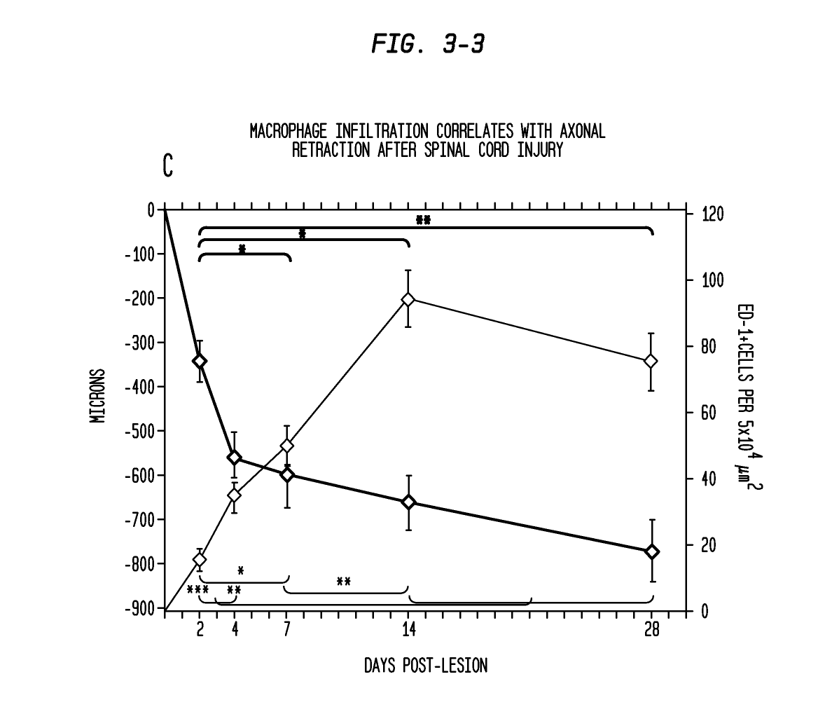

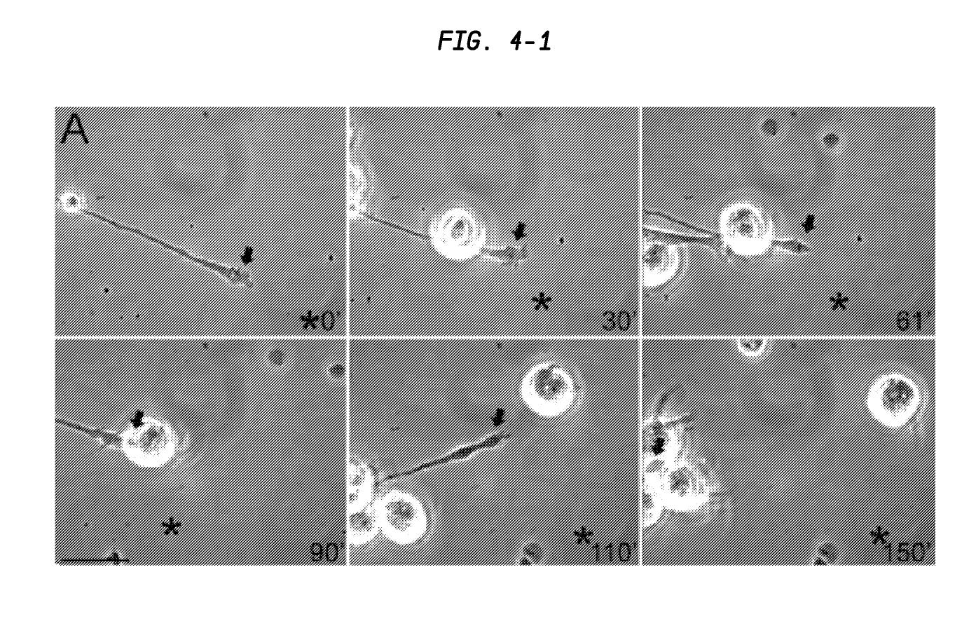

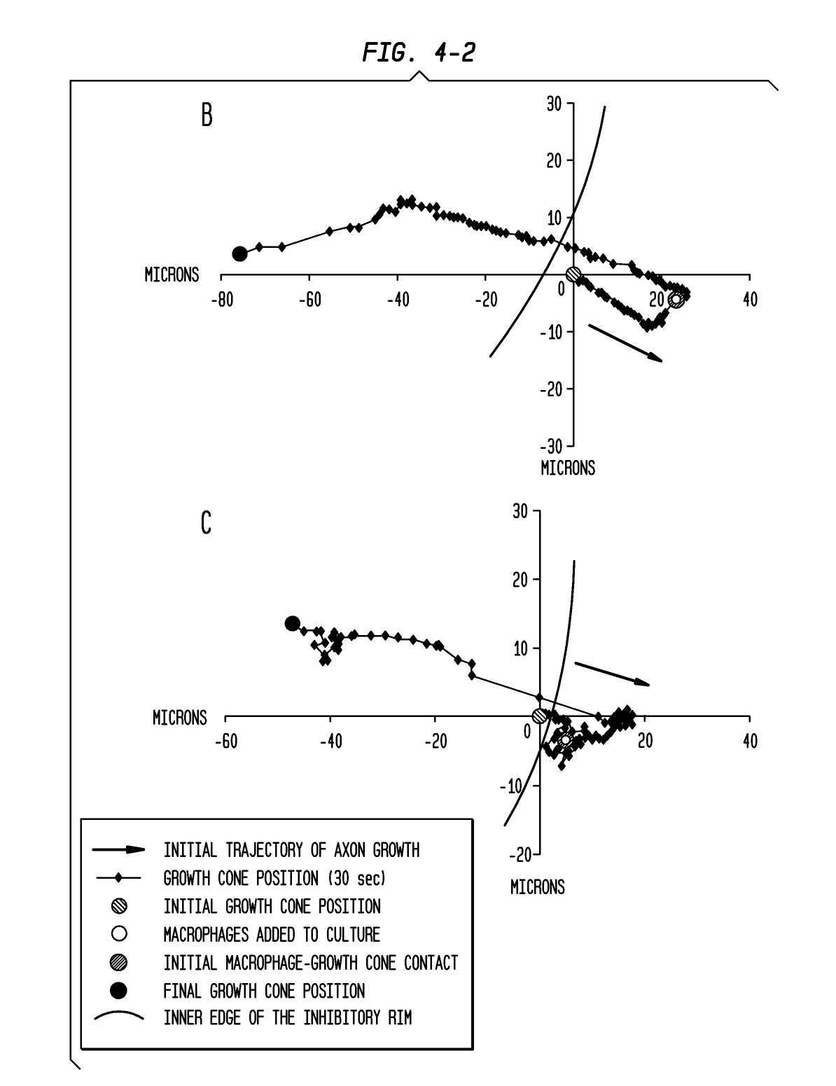

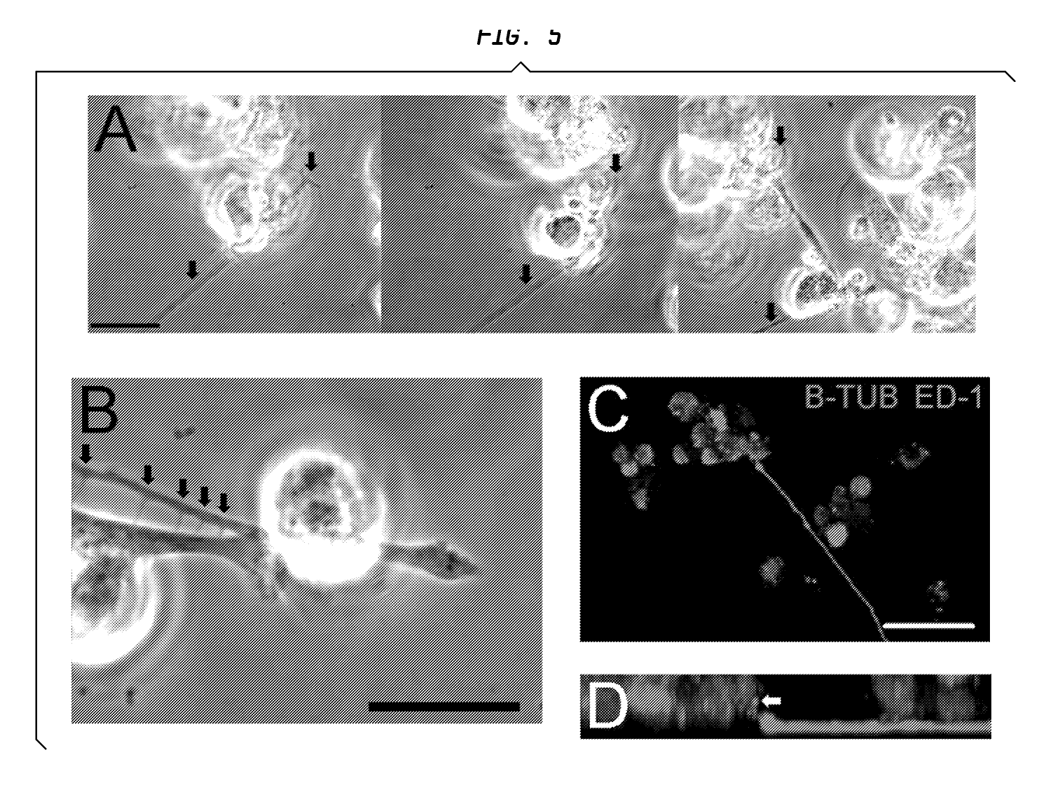

In vivo, a close correlation was found between dystrophic retraction clubs at the ends of severed axons and ED-1⁺ cells following a dorsal column crush spinal cord injury (FIG. 3). The in vitro model of the glial scar (Tom et al., 2004; Steinmetz et al., 2005) was applied to examine the interactions between axons and ED-1⁺ cells in real time. Direct cell-cell contact between dystrophic growth cones and ED-1⁺ macrophages induced a long distance axonal retraction (FIGS. 4, 5). The result of using clodronate liposomes (Popovich et al., 1999) for macrophage depletion in vivo was significant reduction in axonal retraction in the clodronate-treated animals compared to controls. These data indicate that ED-1⁺ cells are directly responsible for retraction of injured spinal cord axons through physical cell-cell interactions.

### Results

### 1. Ascending Dorsal Column Sensory Axons Retract Extensively Following Spinal Cord Injury

It was considered by the inventors that infiltration of activated macrophages could play a direct role in axonal retraction. Within sub-acute and chronic spinal cord lesions a close association was found between activated macrophages and the ends of regenerating axons, allowing for the possibility of direct physical interactions between these two cell types. The extent of sensory axon retraction after dorsal column crush injury was then characterized and correlated with the infiltration of macrophages into the lesion. The dorsal columns of adult female Sprague-Dawley rats were crushed at the level of C8 and a subpopulation of injured neurons were traced via dextran-Texas Red labeling of the sciatic nerve. Spinal cord tissue was harvested at 2, 4, 7, 14, and 28 days post-lesion and the distances between the ends of the labeled fibers and the center of the lesions were measured. By 2 days post-lesion, axons had already retracted an average distance of 343±46.92 um (mean±SD), however, this early retraction was most likely due to intrinsic mechanisms within the neurons themselves (Kerschensteiner et al., 2005). It is important to note that at 2 days post injury the lesion was composed of mainly reactive astrocytes (GFAP⁺) and a few ED-1⁺ cells, which were most likely resident microglia. Between days 2 and 7 post-lesion there was a dramatic increase in the number of ED-1⁺ cells within the lesion, the vast majority of which were most likely infiltrating macrophages (Popovich et al., 1997; Donnelly and Popovich, 2007). The second phase of retraction of ascending sensory fibers within the dorsal columns occurred most rapidly over the first week and then progressively over the next few weeks. By twenty-eight days post-lesion the axons had retracted to an average distance of 774±70.26 um from the lesion epicenter. These data show that the timing of ascending sensory axon retraction corresponds spatiotemporally with the infiltration and accumulation of ED-1⁺ cells within the lesion.

### 2. Depletion of Activated Macrophages Reduces Axonal Retraction In Vivo

The majority of the second phase of axonal retraction in vivo occurs in the ascending dorsal column sensory axons between two and seven days post-lesion corresponding temporally to macrophage infiltration. In order to further implicate macrophages in axonal retraction in vivo animals were treated with clodronate liposomes in order to deplete circulating monocytes/macrophages (van Rooijen et al., 1997; Popovich et al., 1999) Animals received injections of clodronate liposomes every other day starting treatment one day prior to injury to deplete circulating monocytes/macrophages Animals were then assessed for axonal retraction at 2d, 4d, and 7d post-lesion (FIG. 3). Animals that received injections of clodronate liposomes displayed a significant reduction of retraction at 4 d and 7 d post-lesion (402±81.85 um and 439±46.33 um, respectively) as compared to those receiving control liposomes at 4d and 7d post-lesion (586±42.89 um and 806±62.71 um respectively). The reduction in retraction correlated with significantly reduced numbers of ED-1⁺ cells within the lesion in clodronate-treated animals compared to empty liposome controls. Clodronate liposome treatment also resulted in an increase of GFAP⁺ astrocyte processes in the lesion core, correlating with previous observations that macrophage depletion leads to a decrease in cavitation (Popovich et al., 1999). Importantly, there was no difference in the amount of retraction exhibited in the clodronate-treated and control liposome-treated animals at 2 d post-lesion. Macrophage infiltration has not yet occurred at this time, indicating that that the first stage of axonal retraction is macrophage-independent, and perhaps due to endogenous neuronal mechanisms or, potentially, interactions with activated resident microglia. Clodronate-mediated depletion of circulating macrophages/monocytes did prevent axonal retraction normally observed at 4 d and 7 d post-lesion, indicating that this second phase of retraction was caused by the actions of infiltrating macrophages. There was no evidence of significant regeneration (i.e., axonal elongation beyond the center of the lesion) in the clodronate-treated animals.

3. Dystrophic Growth Cones Retract Extensively after Contact with Macrophages in an In Vitro Model of the Glial Scar

The observation of the close association of ED-1⁺ cells with injured axons in vivo suggested that interactions between these cell types may play a role in axonal retraction. The interactions between adult sensory neuron axons and macrophages in an in vitro model of the glial scar were studied. Following a 30-minute period of baseline observation, NR 8383 macrophages were added to the cultures and their interactions with dystrophic axons monitored. Direct cell-cell contacts were frequently observed between dystrophic axons and macrophages. These contacts were of an extended duration and, when coupled with migration of the macrophage, led to dramatic manipulations of the axon that resulted in forceful bending and lifting of the axon from the substrate. It was evident that strong, long-lasting adhesions could be made between the two cell types since lengthy trailing processes connecting the two cells often remained after the macrophages migrated away from the axons. However, macrophage-induced retraction did not permanently prevent re-growth of the axon, as some axons that lost macrophage contact following retraction were able to extend until they again became dystrophic. Direct cell-cell contact between these two cell types eventually led to extensive retraction of the axon 100% of the time. Therefore, macrophage contact induced retraction of dystrophic axons in an in vitro model of the glial scar.

### 4. Direct Physical Cell-Cell Interactions are Required for Macrophage-Induced Retraction

There were numerous instances in which macrophages were observed to migrate very close to, but not touch, dystrophic axons and axonal retraction was not observed in these cases. To determine whether physical interactions between axons and macrophages were required to induce axonal retraction or if macrophage-derived factors were sufficient, macrophages were treated with trypsin to remove extracellular proteins prior to their addition to the DRG cultures. Pre-treatment of macrophages with trypsin still allowed for extensive macrophage mobility and multiple collisions with axons. However, the treatment completely prevented the macrophages from physically tethering to the axons, and consequently no retraction was observed in the absence of long lasting direct cell-cell contacts. It was possible, however, that macrophages were secreting a factor(s) that induced retraction. To test this hypothesis macrophage-conditioned media was added to dystrophic axons in vitro. Macrophage-conditioned media did not induce retraction. Therefore, the mere presence of macrophages or their secreted factors in the vicinity of axons were not capable of inducing axonal retraction in the absence of physical interactions with dystrophic axons.

### 5. Role of the Substrate in Macrophage-Induced Retraction

To determine whether the substrate plays a role in axonal retraction, adult sensory neurons were cultured on a uniform growth-promoting laminin substrate that does not produce dystrophic growth cones. Growth cones on laminin were flattened with numerous filopodia and lamellapodia and overall axon extension occurred at a constant rate. When macrophages were added to these cultures, direct cell-cell contact with axons was observed. However, these contacts were transient and quickly broken, not as extensive, and did not result in axonal retraction. Remnants of membrane contact points were reabsorbed rapidly back into the neurons, and the growth cones continued to extend across the substrate unimpeded. Therefore, macrophage-induced axonal retraction was substrate-dependent; neurons in an active growth state on the permissive substrate laminin were not susceptible to macrophage contact unlike those in a state of dystrophy induced by a CSPG gradient.

6. Activated Primary Macrophages also Induce Axonal Retraction

A further issue was if primary macrophages interacted with dystrophic axons in the same manner as the NR 8383 macrophage cell line in vitro. Progenitor cells from the bone marrow of adult Sprague-Dawley rats were harvested and differentiated into macrophages in vitro, yielding a culture of greater than 80% ED-1⁺ cells. This particular population of macrophages has been shown to retain the phenotypic, morphological and functional characteristics of macrophages found in spinal cord lesions unlike populations harvested from other bodily sources (Longbrake et al., 2007). The ability of primary macrophages to induce axonal retraction was then assayed. Un-stimulated primary macrophages were not capable of inducing retraction. When added to the spot gradient neuronal culture, these macrophages adhered to the substrate but were not motile, displaying characteristics of macrophages in a resting state. Contacts with axons occurred only when macrophages settled directly onto dystrophic axons. Neither the macrophages nor their cell-cell interactions exhibited any of the physical characteristics previously observed with the cell line macrophages, i.e. no tugging, no signs of physical attachment via cell processes, etc.

It was possible that macrophages must be in an activated state in order to interact with dystrophic axons. Primary macrophages were stimulated with the activating cytokine interferon-gamma in culture prior to addition to the time-lapse culture dishes. While these macrophages exhibited a moderate state of activation and a slightly rounded morphology, they were still not motile and did not form strong attachments with dystrophic axons and, consequently did not induce axonal retraction. The primary macrophages were further stimulated with a combination of interferon-gamma and lipopolysaccharide (LPS) prior to addition to the DRG cultures. These macrophages displayed the morphology and behavior of activated macrophages: rounded, phagocytic shape and highly motile. These activated macrophages induced retraction of dystrophic axons as frequently as cell line macrophages. They displayed vigorous physical interactions with dystrophic axons, resulting in strong adhesions between cells and physical grasping, tugging and lifting of axons from the substrate. Primary macrophages, when in an activated state, induced retraction of dystrophic axons in vitro validating the use of cell line macrophages in this study of axonal retraction in vitro. Therefore, the majority of the experiments were carried out with the NR8383 macrophage cell line because it constituted a pure population of cells that were in a constant state of activation, similar to macrophages found within spinal cord lesions without additional stimulation.

### 7. Activated Microglia are Moderately Capable of Inducing Axonal Retraction In Vitro

While macrophages do not typically invade the injured spinal cord until three days post-lesion, resident microglia within the CNS respond to injury immediately (Watanabe et al., 1999). Microglia within a lesion become activated and phagocytic, much like activated macrophages. A limited number of ED-1⁺ cells was found within the lesion at 2 days post-injury, before typical macrophage infiltration, which were most likely resident microglia. The potential contribution of microglia to axonal retraction was assessed using the in vitro model. Cortical microglia were harvested from P1 Sprague-Dawley rats and matured in vitro prior to their addition to time-lapse cultures. Similar to primary macrophages, primary microglia had to be stimulated with interferon-gamma and LPS to become activated in culture. Un-stimulated microglia failed to adhere to the laminin/aggrecan spot gradient substrate, which prevented them from interacting with dystrophic axons in our model. However, stimulated microglia did adhere and physically interact with axons, inducing retraction 50% of the time, however, the contacts between activated microglia and dystrophic axons were not as strong as those of macrophages. Therefore, microglia activated experimentally can also play a role in the induction of axonal retraction.

### 8. Astrocytes, Another CNS Cell Type, Fail to Induce Axonal Retraction In Vitro

Another question was if the induction of retraction of dystrophic axons in vitro is specific to phagocytic cell types normally found within a lesioned spinal cord and not merely the result of the interactions of dystrophic axons with any other cell type. Astrocytes are an integral component of the glial scar following injury to the CNS. They are present in high numbers and extensively contact regenerating axons. Cortical astrocytes were allowed to mature in vitro before addition to DRG cultures. Astrocytes adhered to the substrate and contacted dystrophic axons extensively. Once bound to the substrate, astrocytes migrated rapidly down the aggrecan gradient, away from the rim. Astrocyte processes spread out over axons, sometimes resulting in lateral displacement of the axon. However, these contacts did not lead to retraction of the contacted axon. Therefore, the induction of retraction was specific to interactions with ED-1⁺ phagocytic cells and not merely physical interactions with any other cell type.

### Materials and Methods

### 1. Dorsal Column Crush Lesion Model

Thirty-three adult female Sprague-Dawley rats (250-300g) were used for in vivo studies. Rats were anesthetized with inhaled isofluorane gas (2%) for all surgical procedures. A T1 laminectomy was performed to expose the dorsal aspect of the C8 spinal cord segment. A durotomy was made bilaterally 0.75 mm from midline with a 30 gauge needle. A dorsal column crush lesion was then made by inserting Dumont #3 jeweler's forceps into the dorsal spinal cord at C8 to a depth of 1.0 mm and squeezing the forceps, holding pressure for ten seconds and repeated two additional times. Completion of the lesion was verified by observation of white matter clearing. The holes in the dura were then covered with gel film. The muscle layers were sutured with 4-0 nylon suture, and the skin was closed with surgical staples. Upon closing of the incision, animals received Marcaine (1.0 mg/kg) subcutaneously along the incision as well as Buprenorphine (0.1 mg/kg) intramuscularly. Post-operatively, animals were kept warm with a heating lamp during recovery from anesthesia and allowed access to food and water ad libitum Animals were killed at 2, 4, 7, 14, or 28 days post-lesion (N=3 per group). All animal procedures were carried out in accordance with the guidelines and protocols of the Animal Resource Center at Case Western Reserve University.

### 2. Macrophage Depletion

Animals received intraperitoneal injections of liposome-encapsulated clodronate or empty liposome control on the day before the dorsal column crush injury and also one day post-lesion for the 2 d time-point, on days land 3 post-lesion for the 4 d timepoint, and on days 1, 3, and 5 post-lesion for the 7 d timepoint (N=3 per group). Clodronate was a gift from Roche Diagnostics GmbH, Mannheim, Germany. Clodronate was encapsulated in liposomes as described earlier (Van Rooijen and Sanders, 1994).

### 3. Axon Labeling

Two days before sacrifice, the dorsal columns were labeled unilaterally with Texas-Red conjugated 3000 MW dextran. Briefly, the sciatic nerve of the right hindlimb was exposed and crushed with Dumont #3 forceps for tens seconds and repeated two additional times. 1.0 uL of 3000 MW dextran-Texas-Red 10% in sterile water was the injected via a Hamilton syringe into the sciatic nerve at the crush site. The muscle layers were closed with 4-0 nylon suture and the skin with surgical staples. Upon closing of the incision, animals received Marcaine (1.0 mg/kg) subcutaneously along the incision as well as Buprenorphine (0.1 mg/kg) intramuscularly. Post-operatively, animals were kept warm with a heating lamp during recovery from anesthesia and allowed access to food and water ad libitum Animals were killed two days following labeling with an overdose of isofluorane and perfused with PBS followed by 4% PFA. Tissue was harvested and post-fixed in 4% PFA and processed for immunohistochemistry.

### 4. Immunohistochemistry

Tissue was post-fixed in 4% PFA overnight and then submersed in 30% sucrose overnight, frozen in OCT mounting media, and cut on a cryostat into 20 um longitudinal sections. Tissue was then stained with anti-GFAP (Accurate Chemical and Scientific Corporation, Westbury, NY), anti-ED-1 (Millipore, Billerica, Mass.) and incubated with Alexafluor-405 or Alexafluor-488 (Invitrogen, Carlsbad, Calif.) respectively, and then imaged on a Zeiss Axiovert 510 laser-scanning confocal microscope.

### 5. In Vivo Axonal Retraction Quantification

Three consecutive sections starting at a depth of 200 um below the dorsal surface of the spinal cord per animal were analyzed per animal to quantify axonal retraction. The lesion center was identified via characteristic GFAP and ED-1 staining patterns and the distance between the end of the labeled axons and the centered using Zeiss LSM 5 Image Browser software. The measurements from all sections from all animals in a group were averaged to yield the average distance of retraction per time point.

The technique utilized to trace injured fibers labels axons located very superficially within the dorsal columns. Also, the numbers of fibers labeled can vary due to the extent of fasciculation of the sciatic nerve at the level at which the tracer is injected. Labeled axons were quantified at only that depth for multiple reasons. This depth consistently contained labeled fibers in all animals, whereas some animals did not have labeled fibers at deeper depths. The linear extent of the lesion increases at deeper levels of the dorsal columns. Therefore axons located deeper within the spinal cord encounter a much larger lesion than those at more superficial levels. Quantification of distances of retraction must occur at similar locations of the lesion to allow for accurate comparison between animals and groups. Quantification of the entire population of labeled axons could lead to skewing of results due to differences in the extent of labeling. Instead, a specific population and location of labeled axons were quantified, so they could be consistently examined and accurately quantified in all animals.

### 6. DRG Dissociation

DRGs were harvested as previously described (Tom et al., 2004; Davies et al., 1999). Briefly, DRGs were dissected out of adult female Sprague-Dawley rats (Zivic Miller, Harlan). Both the central and peripheral roots were removed and ganglia incubated in a solution of Collagenase II (200 U/mL, Worthington) and Dispase II (2.5 U/mL, Roche) in HBSS. The digested DRGs were rinsed and gently triturated in fresh HBSS-CMF three times followed by low speed centrifugation. The dissociated DRGs were then resuspended in Neurobasal-A media supplemented with B-27, Glutamax, and Penicillin/Streptamycin (all from Invitrogen) and counted. DRGs were plated on Delta-T dishes (Fisher,) at a density of 3,000 cells/mL for a total of 6,000 cells/dish.

### 7. Time-Lapse Dish Preparation

Delta-T cell culture dishes (Fisher, Pittsburgh, Pa.) were prepared similarly to Tom et al., 2004. Briefly, a single hole was drilled through the upper half of each dish with a number 2 bit to create a port for the addition of cells, enzymes, inhibitors, etc. to the cultures during time-lapse microscopy. Dishes were then rinsed with sterile water and coated with poly-1-lysine (0.1 mg/mL, Sigma) overnight at room temperature, rinsed with sterile water and allowed to dry. Aggrecan gradient spots were created by pipetting 2.0 uL of aggrecan solution (2.0 mg/mL, Sigma in HBSS-CMF, Invitrogen) onto the culture surface and allowed to dry. Six spots were placed per dish. After the aggrecan spots dried completely, the entire surface of the dish was bathed in laminin solution (10 ug/mL, BTI, Stoughton, Mass.) in HBSS-CMF for three hours at 37 degrees Celsius. The laminin bath was then removed immediately before plating of cells. Dishes containing a laminin only substrate were prepared as above with only the laminin bath and no aggrecan. The concentrations of substrates used here differ from those used by Tom et al., 2004. The clarity of the microscopy can be improved by removing the nitrocellulose from the dish preparation protocol. However, to compensate for the difference in substrate binding to the dish surface, the concentrations of the substrates used was recalibrated to those listed above.

Following time-lapse imaging, DRGs were fixed in 4% PFA and immunostained with anti B-tubulin-type III (1:500; Sigma, St. Louis, Mo.) and anti-chondroitin sulfate (CS-56, 1:500, Sigma).

### 8. Cell Line Macrophage Cultures

NR8383 cells (ATCC # CRL-2192), an adult Sprague-Dawley alveolar macrophage cell line were cultured as described by Yin et al. (2003). Briefly, cells were cultured in uncoated tissue culture flasks (Corning) in F-12K media (Invitrogen) supplemented with 15% FBS, Glutamax, Penn/Strep (Invitrogen), and sodium bicarbonate (Sigma) and fed two to three times per week. This cell line formed a mixed culture of adherent and suspended cells and was passed by collecting and replating floating cells at the time of feeding. To prepare the cell line macrophages for time-lapse microscopy experiments, cells were harvested with 0.5% trypsin/EDTA (Sigma) washed three times with serum-free F-12K, and plated in uncoated tissue culture flasks at a density of 1.0×10⁶/mL in serum free F-12K. Prior to use in time-lapse experiments the following day, the cultured cell line macrophages were harvested with EDTA and a cell scraper and resuspended in Neurobasal-A supplemented as above with the addition of HEPES (50 uM, Sigma) at a density of 2.5×10⁵/70 uL.

### 9. Primary Bone Marrow-Derived Macrophage Cultures

Bone marrow progenitor cells were harvested based on previously established protocol (Tobian et al., 2004). Briefly, femurs were removed from adult female Sprague-Dawley rats (225-275g, Harlan). The ends of the femurs were removed, a syringe containing cold DMEM supplemented with 10% FBS, Glutamax, Penn/Strep, beta-mercaptoethanol, and HEPES (Invitrogen) (D10F) was inserted into the femur and the bone marrow was flushed out and collected. The resulting cell mixture was then passed through a 70 micron filter and centrifuged. Supernatant was then removed, the resulting cell pellet resuspended in AKT lysing buffer (BioWhitacre) to lyse red blood cells, and centrifuged. The supernatant was removed and the pellet containing bone marrow precursor cells was resuspended and plated in DMEM above additionally supplemented with 20% LADMAC cell line-conditioned media (generous gift of Dr. Clifford Harding) to induce differentiation into macrophages. Cells were fed on days 5, 7, 9, and harvested for experimentation on day 10 in culture. One day prior to time-lapse experiments, primary macrophages were harvested with trypsin/EDTA, washed three times with D10F, and plated in uncoated petri dishes (Falcon) in D10F at a density of 1.0×10⁶/mL. The following day, the primary macrophages were harvested with EDTA and a cell scraper and resuspended in Neurobasal-A plus HEPES at a density of 5.0×10⁵/70 uL for time-lapse microscopy experiments.

### 10. Cortical Astrocyte Preparation

Cortical astrocytes were collected by removing the cortices of a P0-P1 rat, finely mincing and then treating with 0.5% trypsin in EDTA. Cells were seeded in DMEM/F12 (Invitrogen) with 10% FBS (Sigma) and 2 mM Glutamax on T75 flasks coated with poly-L-lysine and shaken after 4 hours to remove non-adherent cells. Astrocytes were matured in culture for at least 28 days. Astrocytes were harvested with EDTA and a cell scraper and resuspended in Neurobasal-A plus HEPES at a density of 5.0×10⁵/70 uL for time-lapse microscopy experiments.

### 11. Cortical Microglia Preparation

Cortical microglia were collected by removing the cortices of a P0-P1 rat, finely mincing and then treating with 0.5% trypsin in EDTA. Cells were plated in DMEM/F12 (Invitrogen) with 20% FBS (Sigma) and 2 mM Glutamax on T75 flasks coated with poly-L-lysine for 5-7 days. One day prior to time-lapse experiments, flasks were agitated to remove less adherent cells and these cells were plated in uncoated petri dishes (Falcon) in D10F at a density of 1.0×10⁶/mL. The following day, the primary microglia were harvested with EDTA and a cell scraper and resuspended in Neurobasal-A plus HEPES at a density of 5.0×10⁵/70 uL for time-lapse microscopy experiments.

### 12. Time-Lapse Microscopy Studies

DRG neurons were incubated at 37° C. for 48 hours prior to time-lapse imaging. Neurobasal-A media with HEPES (50 uM, Sigma) was added to the culture prior to transfer to a heated stage apparatus. Time-lapse images were acquired every 30 seconds for 3 hours with a Zeiss Axiovert 405M microscope using a 100× oil-immersion objective. Growth cones were chosen that extended straight into the spot rim and had characteristic dystrophic morphology. Neurons were observed for 30 minutes to determine baseline behavior before the addition of additional cell types (N=6 for all groups except primary macrophage, N=3). Growth cones were observed for 150 minutes after cell addition. We tracked extension/retraction, and rate of growth with Metamorph software.

### 13. Statistical Analysis

Data were analyzed by One- or Two-way ANOVA or General linear model, where appropriate, and Tukey post-hoc test with Minitab 15 software.

### Discussion

The results show for the first time conclusive evidence that macrophages induce retraction of dystrophic adult axons through direct physical contact. The induction of retraction was dependent upon both the growth state of the neurons and the activation state of the macrophages. Primary bone marrow-derived macrophages required stimulation with interferon-gamma and LPS to reach a state of activation similar to the cell line macrophages and macrophages within a spinal cord lesion to induce axonal retraction in vitro. This indicates that the behavior of macrophages in vivo is state-dependent and corresponds with previous work showing that macrophage infiltration only correlates with axonal retraction in the presence of myelin degeneration (McPhail et al., 2004). The study shows that adult sensory neurons were only susceptible to macrophage-induced retraction when they were in a dystrophic state of stalled growth induced by a gradient of inhibitory CSPG. Adult neurons in an active state of growth on a uniform laminin substrate rapidly broke contacts with macrophages and did not retract.

The induction of retraction potentially involves multiple intrinsic and extrinsic mechanisms. The study shows that retraction did not occur without direct cell-cell contact between macrophages and dystrophic neurons. The addition of trypsin-treated macrophages or macrophage-conditioned media alone was insufficient to induce retraction. Macrophages did not have to contact dystrophic axons specifically at the growth cone in order to induce retraction. It is possible that contact with activated macrophages may trigger signaling pathways within the axon distant from its dystrophic ending.

There are several candidate binding partners by which macrophages and neurons may physically identify to one another and interact. Macrophages may use alphav and beta1 integrin receptors to recognize and bind to axonal vitronectin (Sobel et al., 1995) and macrophage adhesion to degenerating peripheral nerve is partially attenuated by blocking beta1 integrin (Brown et al., 1997). Following injury to the optic nerve, axons express ephrinB3, which is recognized by the EphB3 receptor present on macrophages (Liu et al., 2006). Sialoadhesin, a macrophage-specific receptor for sialic acid, is present on neuronal cell membranes (Kelm et al., 1994; Tang et al., 1997). Macrophages also recognize phosphatidylserine exposed on the outer membrane surface of cells undergoing apoptosis (De et al., 2002; De Simone et al., 2004) which may flag injured neurons for endocytosis. Additionally, fractalkine is a chemokine expressed predominantly on the surface of CNS neurons, while its receptor, CX3CR1, is found on macrophages (Zujovic et al., 2000; Umehara et al., 2001). Further studies must be done to determine which, if any, of these molecules are expressed or upregulated on the surfaces of dystrophic adult neurons targeting them for macrophage recognition.

This study confirms that the ascending dorsal column pathway undergoes retraction (Borgens et al., 1986; McPhail et al., 2004; Stirling et al., 2004; Baker and Hagg, 2005; Baker et al., 2007) and the timing corresponds to the infiltration of macrophages. Axonal retraction has been examined in other pathways within the spinal cord including the descending cortical spinal tract (Fishman and Kelley, 1984; Iizuka et al., 1987; Hill et al., 2001; Seif et al., 2007), bulbospinal tract (Houle and Jin, 2001), and rubrospinal tract (Schwartz et al., 2005; Cao et al., 2007). It is important to consider that there are two distinct phases of axonal retraction. A recent study which imaged axotomized ascending sensory axons of the adult mouse in vivo showed axonal retraction of about 300 um within the first few hours of injury followed by axon stabilization for the first three days post-lesion (Kerschensteiner et al., 2005). Therefore the focus of this study was on the second, later phase of axonal retraction that is due to activated macrophages and not to the intrinsic properties of the neurons.

This study shows that treatment with clodronate liposomes prior to injury and throughout the normal retraction period prevented the second phase of axonal retraction between two and seven days post-lesion. Previous work has shown that clodronate liposome-mediated macrophage depletion results in reduced lesion volume and increased neuronal survival (Popovich et al., 1999; van Rooijen and van Kesteren-Hendrikx, 2002). While these authors stress the axon regenerative effect of macrophage depletion, the data in this study suggest that the increased axonal content of the lesions of clodronate liposome-treated animals may be due, at least in part, to an attenuation of axonal retraction.

### Example III

### Stem Cells can Prevent Adhesion of Activated Macrophages to DRGs

### Results

### 1. Model

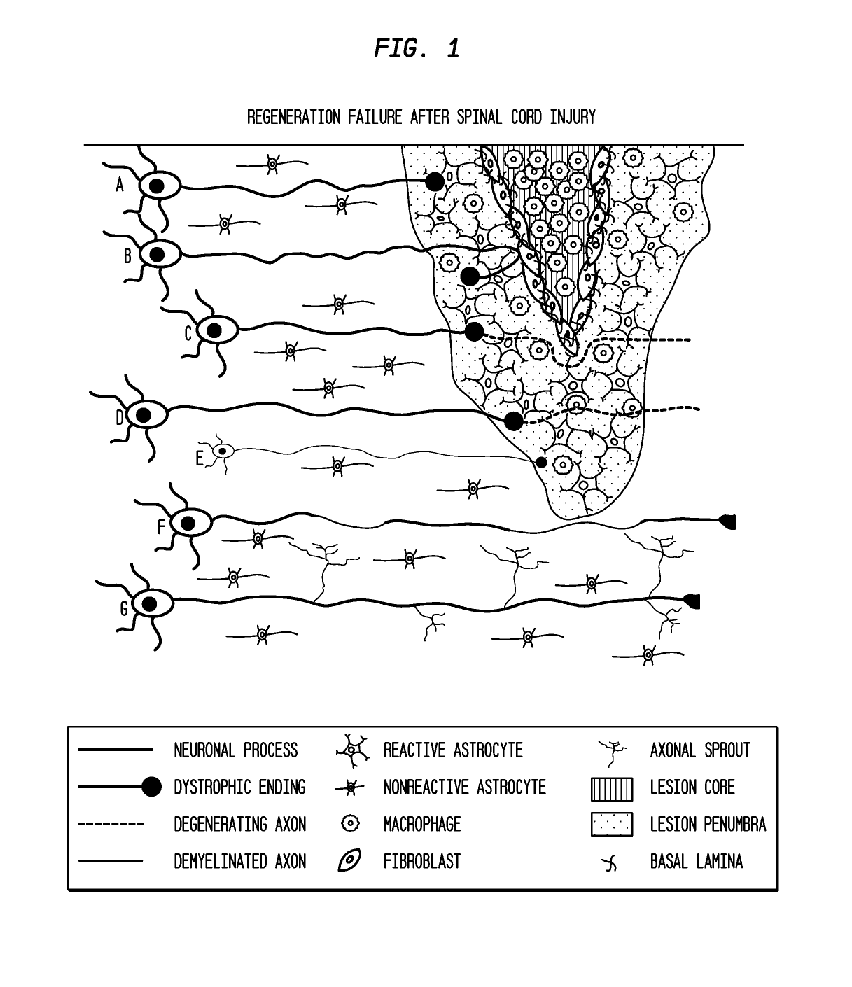

Following dorsal column crush injury, regenerating axons encounter macrophages and microglia and form dystrophic endings. This is shown schematically in FIG. 1. Previous work from the inventors' laboratory has shown that macrophage infiltration is correlated with axonal dieback following dorsal column crush injury (FIGS. 2 and 3). After characterizing the extent of axonal dieback of the ascending dorsal column sensory axons following injury, the inventors established an in vitro model of dieback, which can be used to evaluate various treatment strategies. The in vitro assay consists of cultured adult DRG neurons on a substrate of opposing gradients of the growth-promoting protein laminin and the potently inhibitory chondroitin sulfate proteoglycan aggrecan (Tom et al., 2004). This spot gradient is sufficient to stall axonal growth and induce the formation of dystrophic growth cones like those observed in the injured spinal cord.

Time-lapse microscopy allowed the inventors to closely examine growth cone dynamics, such as the number of filopodia, extent of lamellapodia, and number of vesicles in the dystrophic endings. Direct cell-cell contacts were frequently observed between dystrophic axons and macrophages leading to extensive retraction of the axon (FIGS. 4 and 5). Direct cell contact was necessary to induce retraction, as neither macrophage-conditioned media, nor the presence of macrophages near dystrophic axons resulted in retraction. Therefore, the inventors hypothesized that depletion or modulation of activated macrophages may be a potential therapeutic target in spinal cord injury.

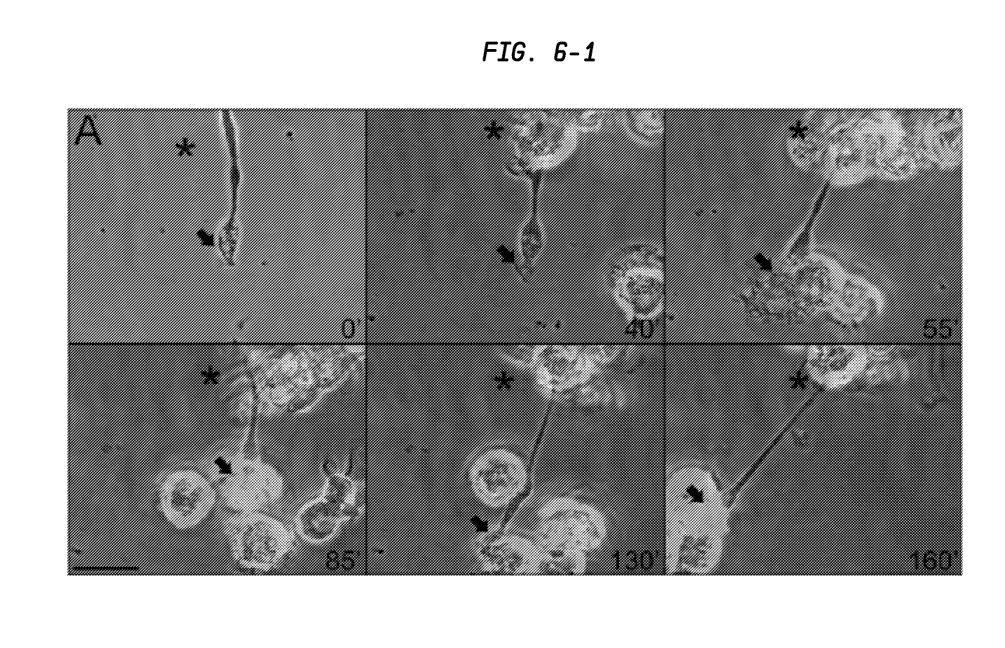

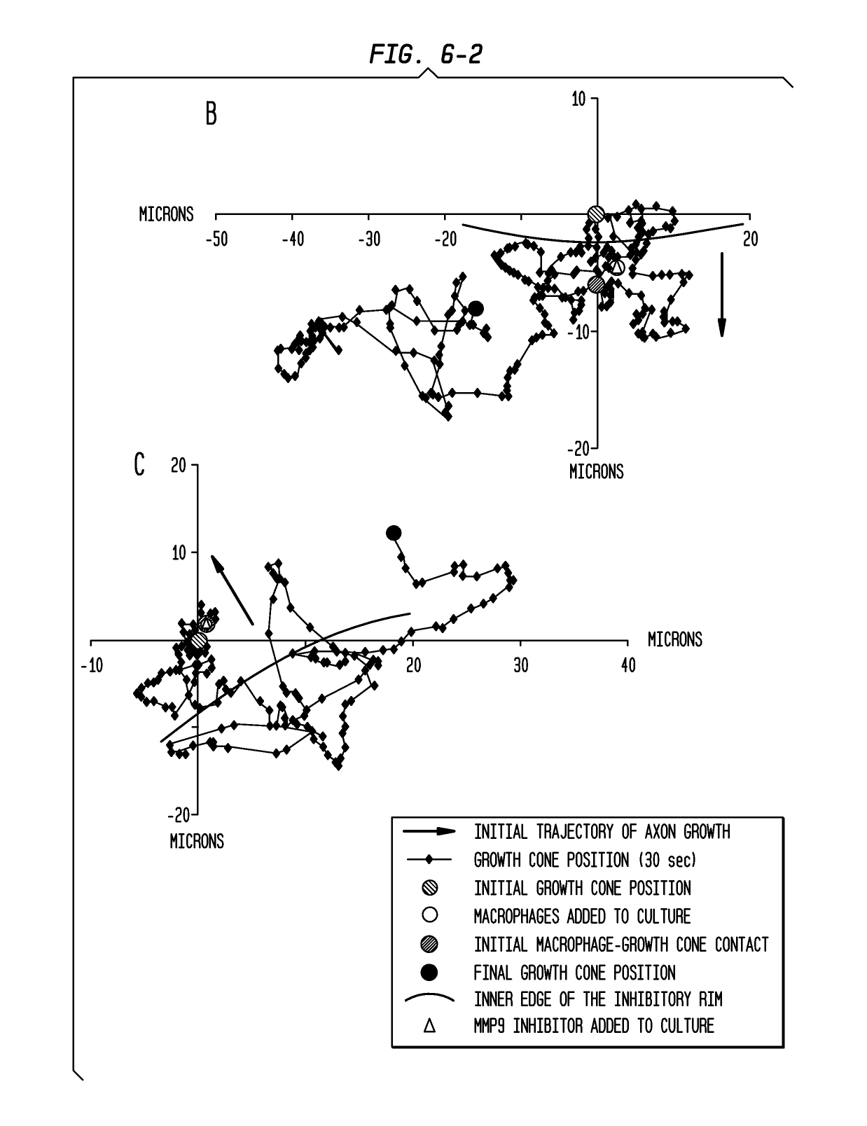

The inventors have elucidated the mechanism by which macrophage-neuron interactions result in dieback. Macrophages are known to secrete a variety of proteases, which aid in the breakdown and clearance of debris, and the inventors have shown that macrophages express and secrete MMP-9. They hypothesized that a protease could be responsible for locally dislodging a dystrophic axon from the substrate causing it to retract. One class of proteases expressed by macrophages is the matrix metalloproteinase (MMP). MMPs have already been implicated in regeneration failure in the CNS as transgenic mice lacking certain MMPs exhibit enhanced axonal regrowth following injury, as do animals treated with the general MMP inhibitor, GM6001. GM6001, which acts as a zinc chelator at MMP active sites, was applied to the timelapse dish at the time of macrophage addition. Treatment with GM6001 or a specific MMP-9 inhibitor (FIG. 6) in the in vitro model prevented the retraction of dystrophic growth cones after direct cell-cell contact with macrophages, while a specific MMP-2 inhibitor did not. GM6001 and the specific MMP-9 inhibitor did not prevent the direct cell-cell contact between macrophages and dystrophic axons. Thus, MMPs, and MMP-9 are implicated as playing a role in axonal dieback.

### 2. MAPCs Prevent Macrophage-Mediated Axonal Dieback In Vitro

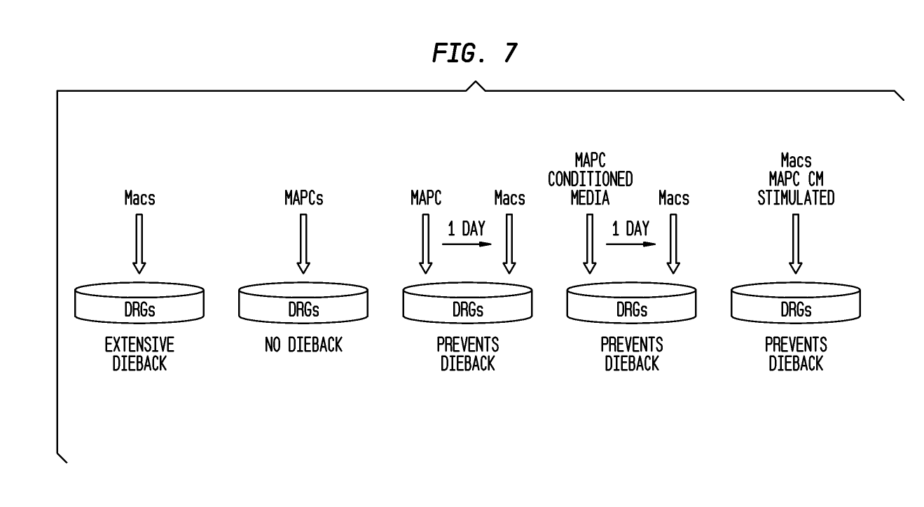

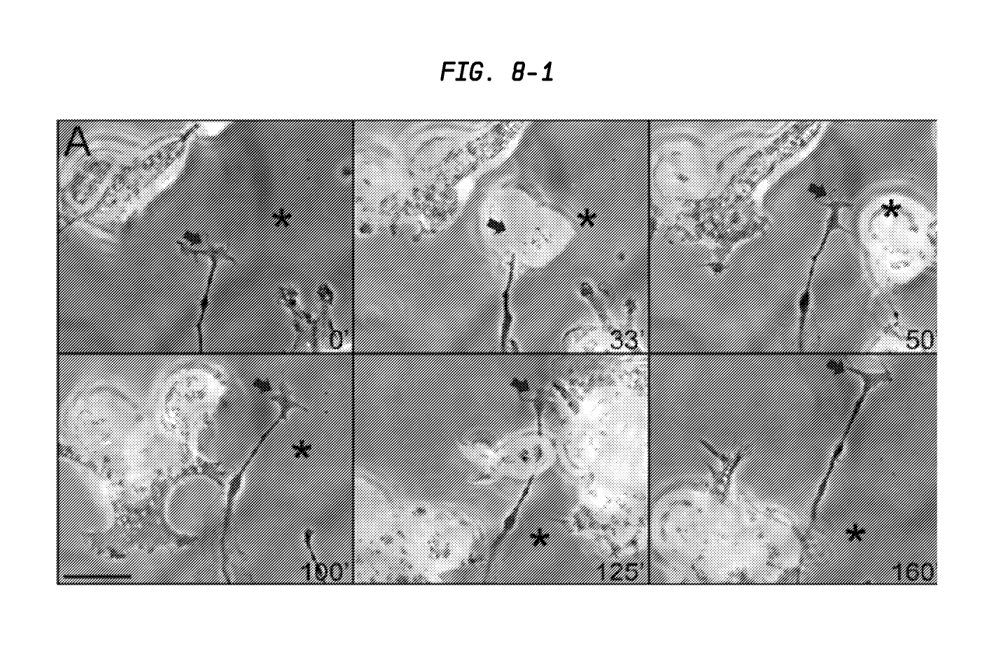

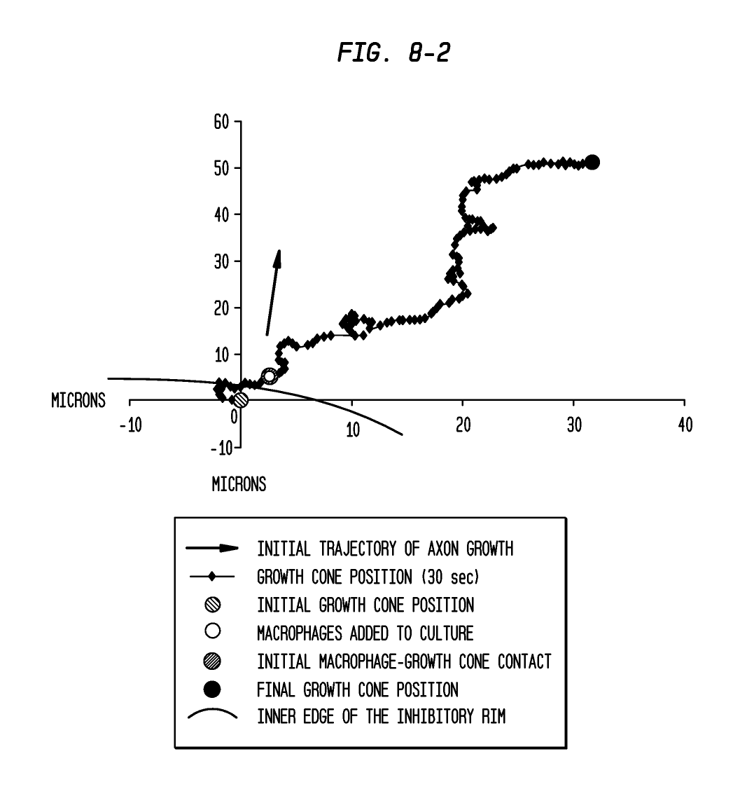

FIG. 7 shows a schematic representation of the experimental design to determine if MAPCs could modulate the inhibitory effects of macrophages. MAPCs were added to 1 DIV DRG spot cultures and incubated for an additional day. Growth cone morphology of these cocultured neurons was quite different from dystrophic growth cones typically found on the spot. These growth cones were increasingly motile, flattened and had extensive lamellapodia. Macrophages contacted the growth cone and axon, but these contacts were often transient, and 5 out of 6 axons imaged did not undergo the characteristic macrophage-mediated retraction (FIG. 8).

This result could be to due to neurostimulatory or immunomodulatory effects of the MAPCs, or both. To address this question, a series of conditioned-media experiments were performed. Dissociated DRG neurons were treated for 24 hours with MAPC-conditioned media and compared with media controls to assess the longest neurite. No significant difference was seen in the length of neurites between groups, suggesting that the effect might not be entirely neurostimulatory.

Direct addition of MAPC-conditioned media to the timelapse dish resulted in a change in growth cone morphology, from a dystrophic, stalled state, to a motile, flattened state. Macrophages still contacted these axons, but contacts were generally transient and generally did not result in axonal retraction. Macrophages pretreated with MAPC-conditioned media also contacted axons on the spot, but did not cause retraction (FIGS. 9-12). It is possible that MAPCs act on macrophages to alter their receptor expression, response to injured cells, or secretion of MMP-9.

### 3. MAPCs Decrease the Extent of Axonal Dieback Following Dorsal Column Crush Injury

The immune-modulating effect of MAPCs on axonal dieback in vivo was investigated using a dorsal column crush model of spinal cord injury. The most dramatic phase of axonal dieback occurs between two and four days post-lesion, which correlates spatiotemporally with the infiltration of activated macrophages into the lesion. It was possible that MAPCs would modulate activated macrophages within the lesion in such a way as to reduce the amount of axonal dieback. Therefore, MAPCs were transplanted into the spinal cord immediately following injury and the extent of axonal dieback was measured at two and four days post-lesion. The MAPCs were transplanted approximately 500 microns caudal to the lesion and 500 microns lateral to the midline. This location was chosen in order to place the MAPCs close to the ends of the injured axons, to minimize further disruption of the ascending tract, and to prevent the cells from being displaced from the spinal cord by blood and CSF flow directly at the lesion site.

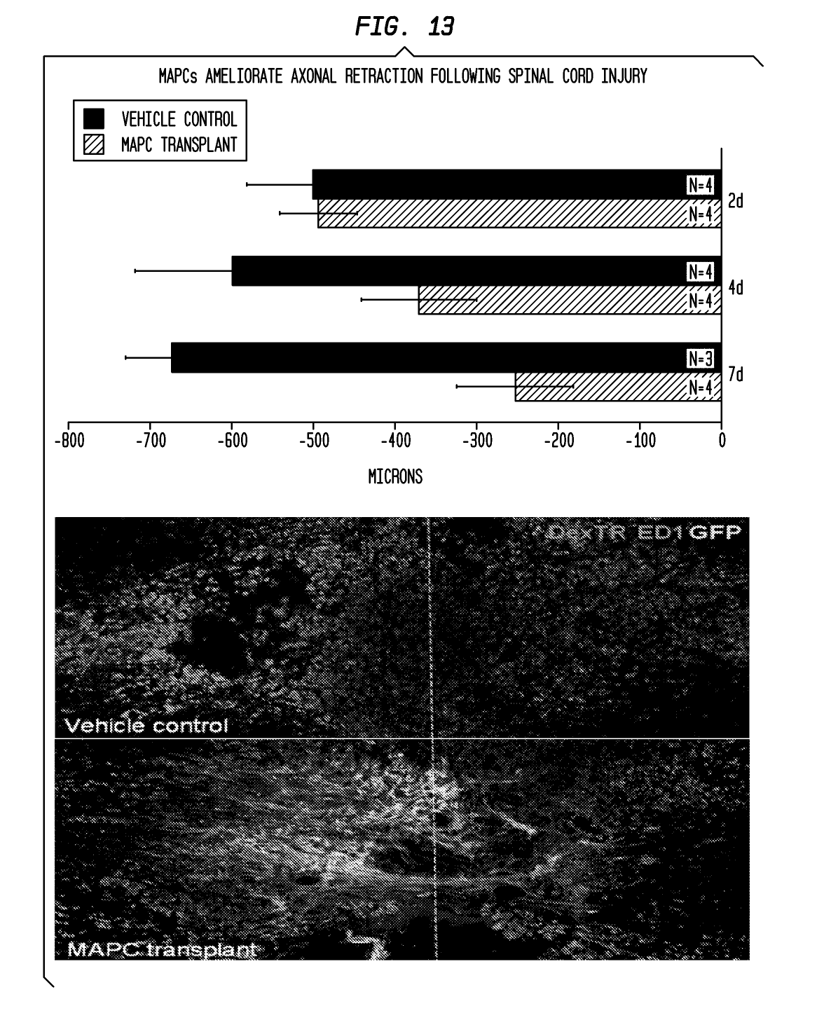

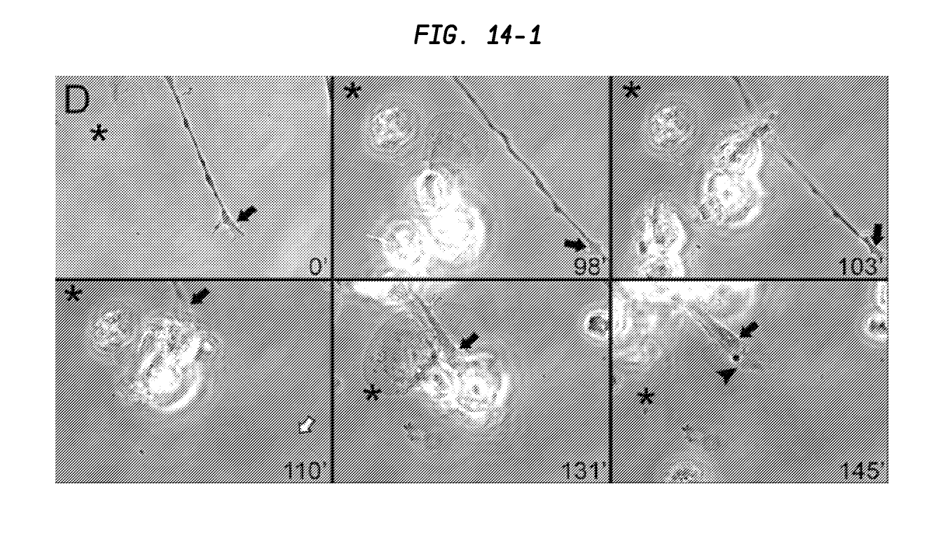

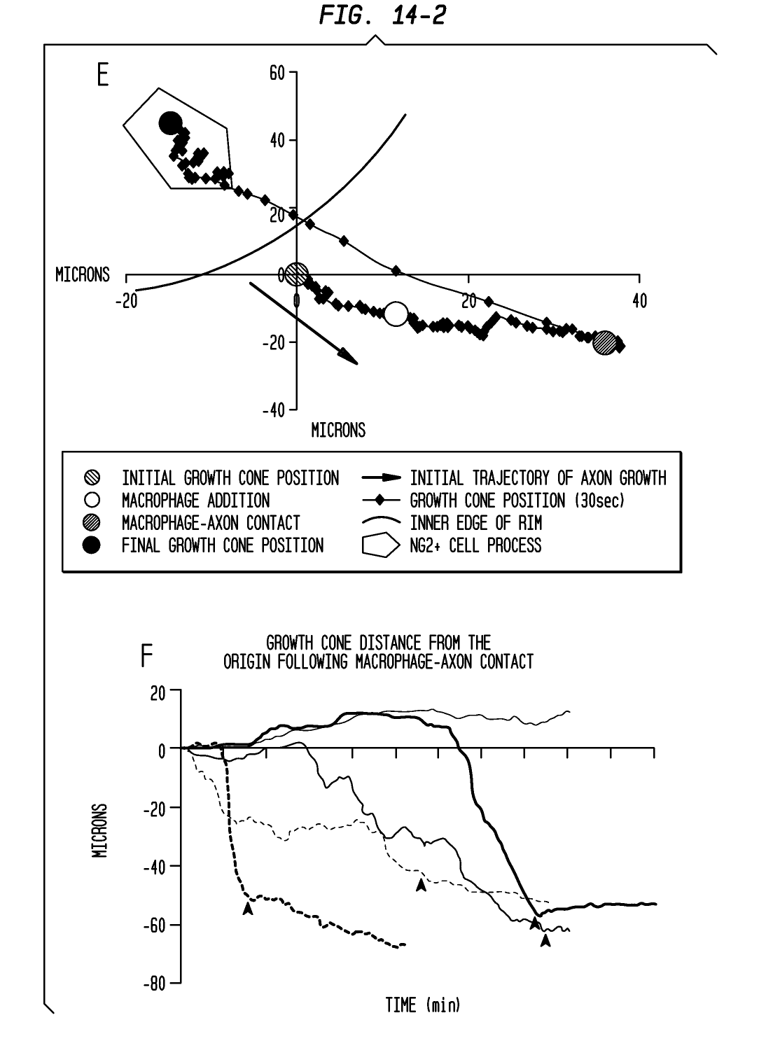

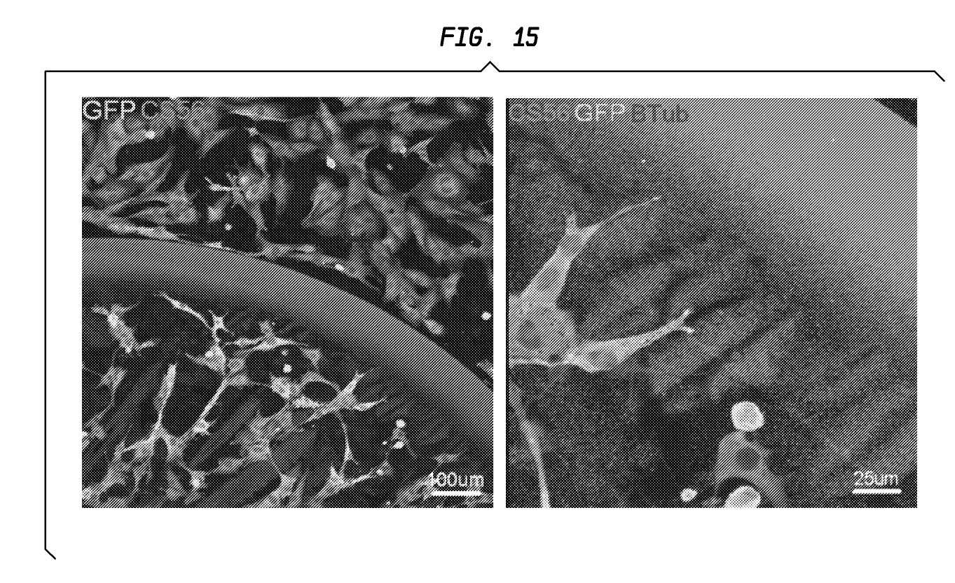

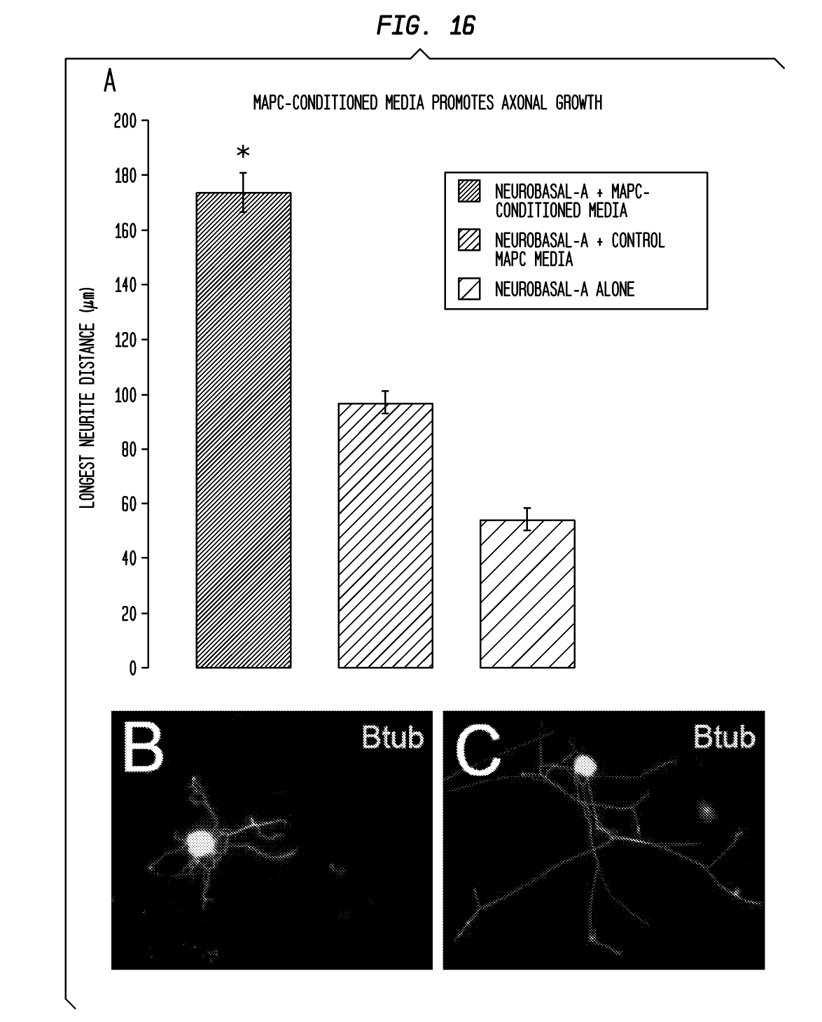

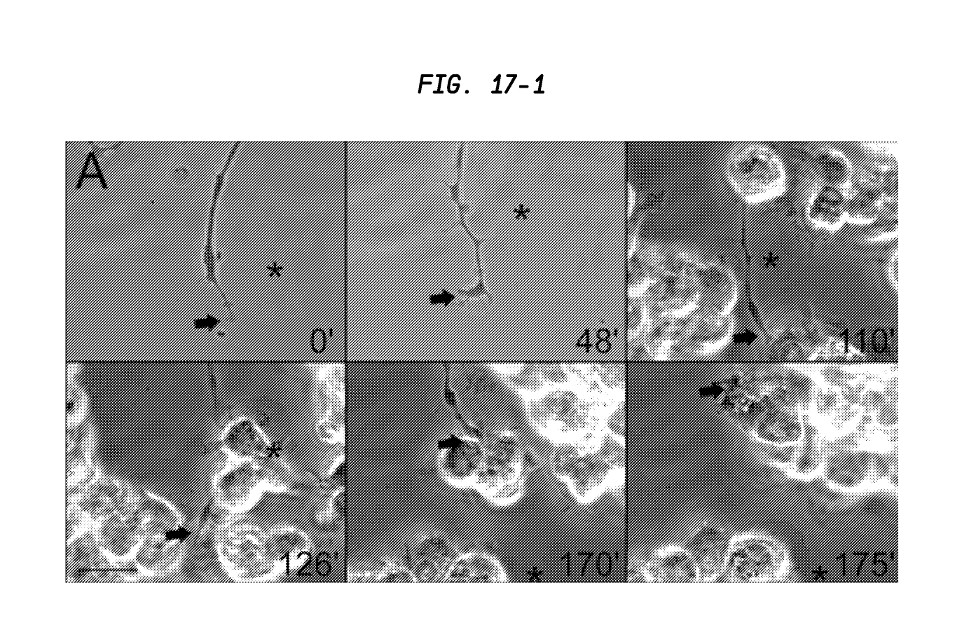

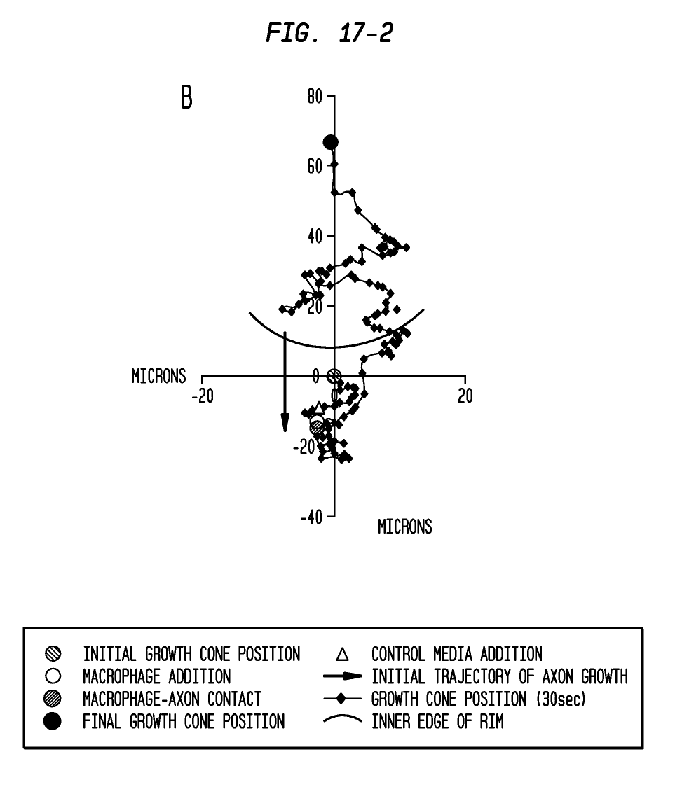

The transplanted MAPCs successfully integrated into the spinal cord tissue as was evidenced by the presence of GFP⁺ cells at the injection site at both two and four days post-lesion. In addition, the MAPCs migrated extensively away from the site of transplantation and were able to occupy the core of the lesion and were also observed to associate with the endings of injured axons. At two days post-lesion, the extent of axonal dieback in MAPC transplanted animals not significant from that of control animals (FIG. 13). MAPC transplantation did not prevent the extent of axonal dieback normally observed at two days post-lesion. However, this initial phase of dieback is most likely due to intrinsic neuronal mechanisms and is not mediated by activated macrophages, as they have not yet infiltrated the lesion at this time.

At four days post-lesion, MAPC transplanted animals showed a significant decrease in the extent of axonal dieback compared to non-injected controls (FIG. 13). The transplantation of MAPCs nearly completely attenuated the dieback normally observed at this time, which this study has shown to be directly caused by the infiltration of activated macrophages. Therefore, the presence of MAPCs within the injured spinal cord is sufficient to reduce macrophage-induced axonal dieback in vivo.

### Example IV

Vimentin/NG2⁺ oligodendrocyte precursor cells in the lesion core start to expand around the time of macrophage infiltration, and the ends of axotomized fibers are associated with this cell population. This suggested that NG2⁺ cells within a CNS lesion serve to stabilize axons, making them an ideal candidate to prevent macrophage-mediated retraction. NG2⁺ glial cells from adult mouse spinal cord were added to DRG cultures after one day in vitro. On day 2, following a 30-minute period of baseline observation, NR 8383 macrophages were added to the timelapse dish and observed for 2.5 additional hours. The presence of NG2⁺ glial cells in coculture with DRGs was not sufficient to prevent macrophage-induced axonal retraction (N=5). In FIG. 14, the axon retracts following macrophage contact and stabilizes on an NG2⁺ glial cell.

### Methods

### 1. DRG Dissociation

DRGs were harvested as previously described (Tom et al., 2004; Davies et al., 1999). Briefly, DRGs were dissected out of adult female Sprague-Dawley rats (Harlan). Both the central and peripheral roots were removed and ganglia incubated in a solution of Collagenase II (200 U/mL, Worthington) and Dispase II (2.5 U/mL, Roche) in HBSS. The digested DRGs were rinsed and gently triturated in fresh HBSS-CMF three times followed by low speed centrifugation. The dissociated DRGs were then resuspended in Neurobasal-A media supplemented with B-27, Glutamax, and Penicillin/Streptamycin (all from Invitrogen) and counted. DRGs were plated on Delta-T dishes (Fisher) at a density of 3,000 cells/mL for a total of 6,000 cells/dish.

### 2. Timelapse Dish Preparation

Delta-T cell culture dishes (Fisher) were prepared similarly to Tom et al., 2004. Briefly, a single hole was drilled through the upper half of each dish with a number 2 bit to create a port for the addition of cells to the cultures during timelapse microscopy. Dishes were then rinsed with sterile water and coated with poly-1-lysine (0.1 mg/mL, Invitrogen) overnight at room temperature. Dishes were then rinsed with sterile water and allowed to dry. Aggrecan gradient spots were created by pipetting 2.0 uL of aggrecan solution (2.0 mg/mL, Sigma in HBSS-CMF, Invitrogen) onto the culture surface and allowed to dry. Six spots were placed per dish. After the aggrecan spots dried completely, the entire surface of the dish was bathed in laminin solution (10 ug/mL, BTI in HBSS-CMF) for three hours at 37 degrees Celsius. The laminin bath was then removed immediately before plating of cells.

### 3. Cell Line Macrophage Cultures

NR8383 cells (ATCC # CRL-2192), an adult Sprague-Dawley alveolar macrophage cell line, were cultured as described in Yin et al., 2003. Briefly, cells were cultured in uncoated tissue culture flasks (Corning) in F-12K media (Invitrogen) supplemented with 15% FBS (Sigma), Glutamax, Penn/Strep (Invitrogen), and sodium bicarbonate (Sigma) and fed two to three times per week. To prepare the cell line macrophages for timelapse microscopy experiments, cells were harvested with trypsin/EDTA (Invitrogen), washed three times, and plated in uncoated tissue culture flasks at a density of 1.0×10⁶/mL in serum-free F-12K. Prior to use in timelapse experiments, the cultured cell line macrophages were harvested with EDTA and a cell scraper and resuspended in Neurobasal-A with the addition of HEPES (50 uM, Sigma) at a density of 2.5×10⁵/70 ul.

### 4. MAPC Cultures

Sprague-Dawley rat MAPC labeled with GFP were grown in rat MAPC media consisting of low glucose DMEM (Invitrogen), 0.4×MCDB-201 medium (Sigma), 1×ITS liquid media supplement (Sigma), 1 mg/ml linoleic acid-albumin (Sigma), 100 U/ml penicillin G sodium/100 μg/ml streptomycin sulfate (Invitrogen), 100 μM 2-β-L-ascorbid acid (Sigma), 100 ng/ml EGF (Sigma), 100 ng/ml PDGF (R&D Systems), 50 nM dexamethasone (Sigma), 1000 U/ml ESGRO (Chemicon), and 2% fetal bovine serum (Hyclone). The cultures were plated on 10 ng/ml fibronectin (Invitrogen) coated 150 cm² tissue culture flasks (Corning) at an initial density of 1000 cell/cm2 and subsequent replating at 200 cells/cm². The cells were maintained in 15 ml of media/flask at 37° C. and 5.0% CO₂ with passaging occurring every 3-4 days using trypsin/EDTA (Invitrogen).

### 5. MAPC-Conditioned Media

Cells were cultured as described above and conditioned media was collected after 48 hours in 50 ml conical tubes (BD Bioscience). The conditioned media was spun down at 400×g for 5 min at 4° C. and the supernatant transferred to a new 50 ml conical tube. The conditioned media was then stored at 4° C.

MAPC-conditioned media was obtained as described above and concentrated 50 fold with an Amicon Microcon Ultracel YM-3 3,000 MWCO centrifugal filter (Millipore, Bedford Mass.).

### 6. MAPC-Conditioned Media-Treated Macrophage

NR8383 rat macrophages were cultured as described above. One day prior to timelapse microscopy experiments, macrophages were harvested with trypsin/EDTA (Invitrogen), washed three times, and plated in uncoated tissue culture flasks at a density of 1.0×10⁶/mL in serum-free F-12K. Twenty uL of the 50-fold concentrated MAPC-conditioned media were added per 1 mL of serum-free F12K media, for a final concentration of 1×. Prior to use in timelapse experiments, the cultured cell line macrophages were harvested with EDTA and a cell scraper and resuspended in Neurobasal-A with the addition of HEPES (50 uM, Sigma) at a density of 2.5×10⁵/70 ul.

### 7. Timelapse Microscopy

DRG neurons were incubated at 37° C. for 48 hours prior to timelapse imaging. Neurobasal-A media with HEPES (50 uM, Sigma) was added to the culture prior to transfer to a heated stage apparatus. Time-lapse images were acquired every 30 seconds for 3 hours with a Zeiss Axiovert 405M microscope using a 100× oil-immersion objective. Growth cones were chosen that extended straight into the spot rim and had characteristic dystrophic morphology for 30 minutes to observe baseline behavior before the addition of cells or conditioned media and then observed for 3 hours.

For cell-addition experiments, cultured rat-derived MAPCs were harvested from tissue culture flasks, washed three times and resuspended in Neurobasal-A media. For coculture experiments, MAPCs (100,000/dish) were added to dorsal root ganglia neuron cultures after 24 hours and incubated at 37° C. for 24 additional hours before timelapse imaging.

For experiments in which MAPC-conditioned media was added to DRG cultures during timelapse imaging, 90 uL of 50×MAPC-CM was added after 30 minutes of baseline growth cone observation.

MAPC-conditioned media-treated macrophages were added to timelapse cultures after 30 minutes of baseline imaging (500,000 cells/dish).

Extension/retraction, rate of growth, turning and branching were analyzed using Metamorph software.

### 8. Immunocytochemisty

Following timelapse imaging, DRGs were fixed in 4% PFA and immunostained with anti-B-tubulin-type III (1:500; Sigma), anti-chondroitin sulfate (CS-56, 1:500, Sigma) and anti-GFP (1:500, Invitrogen).

### 9. Primary Bone Marrow-Derived Macrophage Cultures

Bone marrow progenitor cells were harvested as described in Tobian et al. 2004. Briefly, femurs were removed from adult female Sprague-Dawley rats (Harlan). The ends of the femurs were removed, a syringe containing cold DMEM supplemented with 10% FBS, Glutamax, Penn/Strep, beta-mercaptoethanol, and HEPES (Invitrogen) (D10F) is inserted into the femur and the bone marrow was flushed out and collected. The resulting cell mixture was then passed through a 70 um filter and centrifuged. Supernatant was then removed, the resulting cell pellet resuspended in AKT lysing buffer (BioWhitacre) to lyse red blood cells, and centrifuged. The supernatant was removed and the pellet containing bone marrow precursor cells was resuspended and plated in DMEM above additionally supplemented with 20% LADMAC cell line-conditioned media (Generous gift of Dr. Clifford Harding) to induce differentiation into macrophages. Cells were harvested for experimentation on day 10 in culture. One day prior to timelapse experiments, primary macrophages were harvested with trypsin/EDTA, washed three times with D10F, and plated in uncoated petri dishes (Falcon) in D10F at a density of 1.0×10⁶/ml. The following day, the primary macrophages were harvested with EDTA and a cell scraper and resuspended in Neurobasal-A plus HEPES at a density of 5.0×10⁵/70 ul for timelapse microscopy experiments.

### 10. Dorsal Column Crush Lesion Model

Adult Female Sprague-Dawley rats 250-300g were anesthetized with inhaled isofluorane gas (2%) for all surgical procedures. A T1 laminectomy was performed to expose the dorsal aspect of the C8 spinal cord segment. A durotomy was made bilaterally 0.75 mm from midline with a 30 gauge needle. A dorsal column crush lesion was then made by inserting Dumont #3 jeweler's forceps into the dorsal spinal cord at C8 to a depth of 1.0 mm; squeezing the forceps holding pressure for ten seconds and repeated two additional times. Completion of the lesion was verified by observation of white matter clearing. The holes in the dura were then covered with gel film. The muscle layers were sutured with 4-0 nylon suture and the skin closed with surgical staples. Upon closing of the incision, animals received Marcaine (1.0 mg/kg) subcutaneously along the incision as well as Buprenorphine (0.1 mg/kg) intramuscularly. Post-operatively, animals were kept warm with a heating lamp during recovery from anesthesia and allowed access to food and water ad libitum. Animals were killed at 2, 4, 7, 14, or 28 days post-lesion.

### 11. Cell Transplantation

Cultured rat-derived MAPCs or primary bone marrow-derived macrophages (which had been stimulated with interferon-gamma and LPS for 24 hours) were harvested from tissue culture flasks, washed three times in HBSS-CMF and resuspended in HBSS-CMF at a density of 200,000 cells/uL. Immediately following dorsal column crush injury, 1.0 uL of the cell suspension was injected unilaterally 0.5 mm deep into the right side dorsal columns. The injection site was 0 5 mm lateral to the midline and 0.5 mm caudal to the lesion edge. The cells were injected with forty-four 23.0 mL pulses on 15 second intervals through a pulled glass pipette attached to a Nanoject II (Drummond). The glass pipette was then withdrawn from the spinal cord two minutes after the final injection. Following the transplantation, the injection site was covered with gelfilm, the muscle layers were closed with 4-0 ethicon sutures, and the skin was closed with surgical staples. Post-operatively, animals were kept warm with a heating lamp during recovery from anesthesia and allowed access to food and water ad libitum. Animals were killed two or four days post-lesion.

### 12. Axon Labeling

Two days before sacrifice, the dorsal columns were labeled unilaterally with Texas-Red conjugated 3000 MW dextran. Briefly, the sciatic nerve of the right hindlimb was exposed and crushed three times with Dumont #3 forceps for ten seconds. 1.0 uL of 3000 MW dextran-Texas-Red 10% in sterile water was the injected via a Hamilton syringe into the sciatic nerve at the crush site. The muscle layers were closed with 4-0 nylon suture and the skin with surgical staples. Upon closing of the incision, animals received Marcaine (1.0 mg/kg) subcutaneously along the incision as well as Buprenorphine (0.1 mg/kg) intramuscularly. Post-operatively, animals will be kept warm with a heating lamp during recovery from anesthesia and allowed access to food and water ad libitum. Animals were killed two days following labeling with an overdose of isofluorane and perfused with PBS followed by 4% PFA. Tissue was harvested and post-fixed in 4% PFA and processed for immunohistochemistry.

### 13. Immunohistochemistry

Tissue was post-fixed in 4% PFA overnight and then submersed in 30% sucrose overnight, frozen in OCT mounting media, and cut on a cryostat into 20 um longitudinal sections. Tissue was then stained with anti-GFAP/Alexafluor-405, anti-ED-1/Alexafluor-594 or -633, anti-GFP/Alexafluor-488, and anti-vimentin/Alexafluor-633. And then imaged on a Zeiss Axiovert 510 laser-scanning confocal microscope at 10× magnification.

### 14. Axonal Dieback Quantification

To quantify axonal dieback three consecutive sections per animal were analyzed, starting at a depth of 200 um below the dorsal surface of the spinal cord. The lesion center was identified via characteristic GFAP and/or vimentin staining patterns and then centered using Zeiss LSM 5 Image Browser software. The distance between the ends of 5 labeled axons projecting farthest into the lesion and the lesion center was then measured. The measurements from all sections from all animals in a group were averaged to yield the average distance of dieback per time point.

### Example V

Mesenchymal stem cells can be commercial obtained. For example, Rat Mesenchymal Stem Cell Kit (Millipore Catalog No. SCR026) provides read-to-use primary mesenchymal stem cells isolated from the bone marrow of adult Fisher 344 rats along with a panel of positive and negative selection markers for the characterization of mesenchymal stem cell population. Positive cell markers include antibodies directed again two cell-surface molecules (integrin b1 and CD54) that are present on mesenchymal stem cells. Negative cell markers include antibodies directed against two specific hematopoietic cell surface markers, (CD14, present on leukocytes and CD45, present on monocytes and macrophages) that are not expressed by mesenchymal stem cells. These mesenchymal stem cells were assessed for the ability to reduce retraction and were found to reduce retraction (reduce adhesion) in vitro (glial scar).

### Example VI

MAPC-conditioned media treatment of adult DRGs grown on 5 μg/ml laminin promotes neurite outgrowth. See FIG. 16. The longest axon from each dissociated DRG was measured for the group to which media containing Neurobasal-A and either MAPC-conditioned media, control media, or no additional media were added. All conditions are significant from one another, One-way ANOVA, *p<0.0001. B, 16× image representing the average amount of outgrowth of an untreated DRG neuron. C, 16× image representing the average amount of outgrowth of DRGs pretreated with MAPC-conditioned media.

This result could be to due to neurostimulatory or immunomodulatory effects of the MAPCs, or both. To address this question, a series of conditioned-media experiments were performed. Dissociated DRG neurons were treated for 24 hours with fresh MAPC-conditioned media and compared with media controls. The longest neurite of each DRG was measured. Fresh MAPC-conditioned media significantly increased outgrowth on laminin. This suggests that MAPCs secrete one or more growth factors that have a neurostimulatory effect on adult neurons. Previously frozen MAPC-conditioned media did not have the same effect on neurons, suggesting that the factor was altered or inactivated in the freezing process.

### REFERENCES

Arantes et al., Neuroreport 11:177-181 (2000)
Baker et al., Exp Neurol 205:82-91 (2007)
Baker and Hagg, J Neurotrauma 22:415-428 (2005)
Basch et al., (1983)
Batinic, D., et al., (1990)
Borgens et al., J Comp Neurol 250:157-167 (1986)
Brown et al., Eur J Neurosci 9:2057-2063 (1997)
Busch and Silver, Curr Opin Neurobiol 17:120-127 (2007)
Cai Z. H., et al., (1988)
Cao et al., Neurorehabil Neural Repair (2007)
Chang, P., et al., (1999)
Chang, T. M., (1992)
Davies et al., J Neurosci 19:5810-5822 (1999)
De et al., J Neuropathol Exp Neurol 61:237-244 (2002)
De Simone et al., Mol Neurobio 29:197-212 (2004)
Donnelly and Popovich, Exp Neurol 209:378-388 (2008)
Farr et al., Aplysia J Neurobiol 46:89-96 (2001)
Fayaz and Tator, J Neurosci Methods 102:69-79 (2000)
Fishman and Kelley, Neurology 34:1161-1167 (1984)
Fitch and Silver, J Exp Neurol 148:587-603 (1997)
Fitch et al., J Neurosci 19:8182-8198 (1999)
Grimpe and Silver, J Neurosci 24:1393-1397 (2004)
Hill et al., Exp Neurol 171:153-169 (2001)
Hohlfeld et al., Neurology 68:S58-S63 (2007)
Houle and Jin, Exp Neurol 169:208-217 (2001)
Iizuka et al., J Neurosurg 66:595-603 (1987)
Jones et al., Exp Neurol 182:399-411 (2003)
Kelm et al., Curr Biol 4:965-972 (1994)
Kerschensteiner et al., Nat Med 11:572-577 (2005)
Kwon et al., Proc Natl Acad Sci USA 99:3246-3251 (2002)
Li and Raisman, Exp Neurol 134:102-111 (1995)
Liu et al., J Neurosci 26:3087-3101 (2006)
Longbrake et al., J Neurochem 102:1083-1094 (2007)
Matthew, H. W., et al., (1991)
McKeon et al., J Neurosci 11:3398-3411 (1991)
McPhail et al., Eur J. Neurosci 20:1984-1994 (2004)
Morgenstern et al., Prog Brain Res 137:313-322 (2002)
Muschler, G. F., et al., (1997)
Oudega et al., Eur J. Neurosci 11:2453-2464 (1999)
Prewitt et al., Exp Neurol 148:433-443 (1997)
Popovich et al., J Comp Neurol 377:443-464 (1997)
Popovich and Longbrake, Nat Rev Neurosci 9:481-493 (2008)
Popovich et al., Exp Neurol 158:351-365 (1999)
Raff et al., Science 296:868-871 (2002)
Ramon y Cajal, London: Oxford UP (1928)
Rapalino et al., Nat Med 4:814-821 (1998)
Schwartz et al., Neurosurgery 44:1041-1045 (1999b)
Schwartz et al., Am J Neuroradial 26:7-18 (2005)
Seif et al., J Neurotrauma 24:1513-1528 (2007)
Silver and Miller, Nat Rev Neurosci 5: 146-156 (2004)
Snow et al., Exp Neurol 109:111-130 (1990)
Sobel et al., Exp Neurol 54:202-213 (1995)
Steinmetz et al., J Neurosci 25:8066-8076 (2005)
Stirling et al, J Neurosci 24:2182-2190 (2004)
Tang et al., J Neurosci Res 71; 427-444 (2003)
Tang et al., J Cell Biol 138:1355-1366 (1997)
Tobian et al., J Immunol 172:5277-5286 (2004)
Tom et al., J Neurosci 24:6531-6539 (2004)
Umehara et al., Immunol Cell Biol 79:298-302 (2001)
van Rooijen et al., Trends Biotechnol 15:178-185 (1997)
van Rooijen and van Kesteren-Hendrikx, J Liposome Res 12:81-94 (2002)
van Rooijen and Sanders, J Immuno Methods 174:83-93 (1994)
Watanabe et al., J Neurotrauma 16:255-265 (1999)
Wysocki and Sato, (1978)
Xie and Zheng, Exp Neurol 209:302-312 (2008)
Yanagi, K. et al., (1989)
Yin et al., J. Neurosci 23:2284-2293 (2003)
Yiu and He, Nat Rev Neurosci 7:617-627 (2006)
Zujovic et al., Glia 29:305-315 (2000)

1-13. (canceled)

14. A method for reducing the adhesion of ED-1⁺ cells to dystrophic axons that would result in axonal retraction, said method comprising administering stem cells, or factors secreted therefrom, in sufficient proximity to the dystrophic axons and/or ED-1⁺ cells, for time sufficient, and in sufficient amounts to reduce said adhesion.

15. The method of claim 14 wherein reducing said adhesion of ED-1⁺ cells to dystrophic axons in a subject reduces axonal retraction in said subject.

16. The method of claim 14 wherein reducing said adhesion of ED-1⁺ cells to dystrophic axons reduces axonal retraction in a subject and reduces neural injury that is associated with said axonal retraction in said subject.

17. The method of claim 14 wherein reducing said adhesion of ED-1⁺ cells to dystrophic axons promotes axon regeneration in a subject.

18. The method of claim 14 wherein said ED-1⁺ cells are macrophages and/or microglia.

19. The method of claim 14 wherein the secreted factors are derived from cell culture medium conditioned by culturing the stem cells therein, the factors being in a pharmaceutically-acceptable carrier.

20. The method of claim 14 wherein the stem cell is a non-embryonic stem cell that has the ability to differentiate into cell types of more than one embryonic germ layer and/or express one or more of oct4, telomerase, rex-1, rox-1, sox-2, and SSEA4.

21. The method of claim 14 wherein the stem cell is a tissue-specific stem cell.

22. The method of claim 21 wherein the tissue-specific stem cell is a hematopoietic stem cell, neural stem cell, or mesenchymal stem cell.

23. The method of claim 16 wherein the neuronal injury is the result of spinal cord injury, brain injury, stroke, multiple sclerosis, epilepsy, or neuro-degenerative disease.

24. The method of claim 23, wherein the neuro-degenerative disease Alzheimer's Disease, Parkinson's Disease, amylotropic lateral sclerosis, and Creutzfeldt-Jakob Disease.

25. The method of claim 14 wherein said secreted factors are administered.

26. The method of claim 25 wherein said secreted factors are in medium conditioned by culturing the stem cells therein.
